SEQUENCE LISTING (25 sequences)

SEQ ID NO: 1 - protein, 40 aa, artificial sequence
  This is a functional biological equivalent of GHRH.
HVDAIFTNSYRKVLAQLSARKLLQDILNRQQGERNQEQGA

SEQ ID NO: 2 - protein, 40 aa, artificial sequence
  This is a functional biological equivalent of GHRH.
YIDAIFTNSYRKVLAQLSARKLLQDILNRQQGERNQEQGA

SEQ ID NO: 3 - protein, 40 aa, artificial sequence
  This is a functional biological equivalent of GHRH.
YVDAIFTNSYRKVLAQLSARKLLQDILNRQQGERNQEQGA

SEQ ID NO: 4 - protein, 40 aa, artificial sequence
  This is a functional biological equivalent of GHRH.
YADAIFTNSYRKVLAQLSARKLLQDILNRQQGERNQEQGA

SEQ ID NO: 5 - protein, 40 aa, artificial sequence
  This is the artificial sequence for the (1-44)NH2
YADAIFTNSYRKVLGQLSARKLLQDIMSRQQGERNQEQGA

SEQ ID NO: 6 - protein, 40 aa, artificial sequence
  This is the artificial sequence for GHRH (1-40)OH.
XXDAIFTNSYRKVLXQLSARKLLQDIXXRQQGERNQEQGA

SEQ ID NO: 7 - DNA, 323 bp, artificial sequence
  This is a nucleic acid sequence of a eukaryotic promoter c5-12.
cggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcg

SEQ ID NO: 8 - DNA, 190 bp, artificial sequence
  This is a nucleic acid sequence of a human growth hormone (“hGH”) poly A tail.
gggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtaggg

SEQ ID NO: 9 - DNA, 219 bp, artificial sequence
  This is the cDNA for Porcine growth hormone releasing hormone
atggtgctctgggtgttcttctttgtgatcctcaccctcagcaacagctcccactgctccccacctccccctttgaccctcaggatgcggcggcacgtagatgccatcttcaccaacagctaccggaaggtgctggcccagctgtccgcccgcaagctgctccaggacatcctgaacaggcagcagggagagaggaaccaagagcaaggagcataatga

SEQ ID NO: 10 - protein, 40 aa, artificial sequence; identical to SEQ ID NO: 5
  This is the amino acid sequence for porcine growth hormone releasing hormone.

SEQ ID NO: 11 - DNA, 3534 bp, artificial sequence
  Sequence for the operatively linked components of the HV-GHRH plasmid.
gttgtaaaacgacggccagtgaattgtaatacgactcactatagggcgaattggagctccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccaaggcccaactccccgaaccactcagggtcctgtggacagctcacctagctgccatggtgctctgggtgttcttctttgtgatcctcaccctcagcaacagctcccactgctccccacctccccctttgaccctcaggatgcggcggcacgtagatgccatcttcaccaacagctaccggaaggtgctggcccagctgtccgcccgcaagctgctccaggacatcctgaacaggcagcagggagagaggaaccaagagcaaggagcataatgactgcaggaattcgatatcaagcttatcggggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattcgataccgtcgacctcgagggggggcccggtaccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagcaactgttgggaagggcgatcggtgcgggcctcttcgctattacgccagctggcgaaagggggatgtgctgcaaggcgattaagttgggtaacgccagggttttcccagtcacgac

SEQ ID NO: 12 - DNA, 3534 bp, artificial sequence
  Sequence for the operatively linked components of the TI-GHRH plasmid.
gttgtaaaacgacggccagtgaattgtaatacgactcactatagggcgaattggagctccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccaaggcccaactccccgaaccactcagggtcctgtggacagctcacctagctgccatggtgctctgggtgttcttctttgtgatcctcaccctcagcaacagctcccactgctccccacctccccctttgaccctcaggatgcggcggtatatcgatgccatcttcaccaacagctaccggaaggtgctggcccagctgtccgcccgcaagctgctccaggacatcctgaacaggcagcagggagagaggaaccaagagcaaggagcataatgactgcaggaattcgatatcaagcttatcggggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattcgataccgtcgacctcgagggggggcccggtaccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagcaactgttgggaagggcgatcggtgcgggcctcttcgctattacgccagctggcgaaagggggatgtgctgcaaggcgattaagttgggtaacgccagggttttcccagtcacgac

SEQ ID NO: 13 - DNA, 3534 bp, artificial sequence
  Sequence for the operatively linked components of the TV-GHRH plasmid.
gttgtaaaacgacggccagtgaattgtaatacgactcactatagggcgaattggagctccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccaaggcccaactccccgaaccactcagggtcctgtggacagctcacctagctgccatggtgctctgggtgttcttctttgtgatcctcaccctcagcaacagctcccactgctccccacctccccctttgaccctcaggatgcggcggtatgtagatgccatcttcaccaacagctaccggaaggtgctggcccagctgtccgcccgcaagctgctccaggacatcctgaacaggcagcagggagagaggaaccaagagcaaggagcataatgactgcaggaattcgatatcaagcttatcggggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattcgataccgtcgacctcgagggggggcccggtaccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagcaactgttgggaagggcgatcggtgcgggcctcttcgctattacgccagctggcgaaagggggatgtgctgcaaggcgattaagttgggtaacgccagggttttcccagtcacgac

SEQ ID NO: 14 - DNA, 3534 bp, artificial sequence; identical to SEQ ID NO: 12
  Sequence for the operatively linked components of the 15/27/28 GHRH plasmid.

SEQ ID NO: 15 - DNA, 3534 bp, artificial sequence
  This is the entire plasmid sequence for wildtype GHRH.
gttgtaaaacgacggccagtgaattgtaatacgactcactatagggcgaattggagctccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccaaggcccaactccccgaaccactcagggtcctgtggacagctcacctagctgccatggtgctctgggtgttcttctttgtgatcctcaccctcagcaacagctcccactgctccccacctccccctttgaccctcaggatgcggcggtatgcagatgccatcttcaccaacagctaccggaaggtgctgggccagctgtccgcccgcaagctgctccaggacatcatgagcaggcagcagggagagaggaaccaagagcaaggagcataatgactgcaggaattcgatatcaagcttatcggggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattcgataccgtcgacctcgagggggggcccggtaccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagcaactgttgggaagggcgatcggtgcgggcctcttcgctattacgccagctggcgaaagggggatgtgctgcaaggcgattaagttgggtaacgccagggttttcccagtcacgac

SEQ ID NO: 16 - DNA, 4260 bp, Artificial sequence
  This is the sequence for the pSP-SEAP cDNA construct
ggccgtccgccttcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccccgggctgcaggaattcgatatcaagcttcgaatcgcgaattcgcccaccatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttactctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgta

SEQ ID NO: 17 - DNA, 2710 bp, artificial sequence
  Plasmid vector with optimized analog mGHRH codon sequence.
tgtaatacgactcactatagggcgaattggagctccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagcggatcccaaggcccaactccccgaaccactcagggtcctgtggacagctcacctagctgccatggtgctctgggtgctctttgtgatcctcatcctcaccagcggcagccactgcagcctgcctcccagccctcccttcaggatgcagaggcacgtggacgccatcttcaccaccaactacaggaagctgctgagccagctgtacgccaggaaggtgatccaggacatcatgaacaagcagggcgagaggatccaggagcagagggccaggctgagctgataagcttatcggggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggctcgagggggggcccggtaccagcttttgttccctttagtgagggttaatttcgagcttggtcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggctagcgcttagaagaactcatccagcagacggtagaatgcaatacgttgagagtctggagctgcaataccatacagaaccaggaaacggtcagcccattcaccacccagttcctctgcaatgtcacgggtagccagtgcaatgtcctggtaacggtctgcaacacccagacgaccacagtcaatgaaaccagagaaacgaccattctcaaccatgatgttcggcaggcatgcatcaccatgagtaactaccaggtcctcaccatccggcatacgagctttcagacgtgcaaacagttcagccggtgccagaccctgatgttcctcatccaggtcatcctggtcaaccagacctgcttccatacgggtacgagcacgttcaatacgatgttttgcctggtggtcaaacggacaggtagctgggtccagggtgtgcagacgacgcattgcatcagccatgatagaaactttctctgccggagccaggtgagaagacagcaggtcctgacccggaacttcacccagcagcagccagtcacgaccagcttcagtaactacatccagaactgcagcacacggaacaccagtggttgccagccaagacagacgagctgcttcatcctgcagttcattcagagcaccagacaggtcagttttaacaaacagaactggacgaccctgtgcagacagacggaaaacagctgcatcagagcaaccaatggtctgctgtgcccagtcataaccaaacagacgttcaacccaggctgccggagaacctgcatgcagaccatcctgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagcaactgttgggaagggcgatcg

SEQ ID NO: 18 - DNA, 2713 bp, artificial sequence
  Plasmid vector with optimized analog rGHRH codon sequence.
tgtaatacgactcactatagggcgaattggagctccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagcggatcccaaggcccaactccccgaaccactcagggtcctgtggacagctcacctagctgccatggccctgtgggtgttcttcgtgctgctgaccctgaccagcggaagccactgcagcctgcctcccagccctcccttcagggtgcgccggcacgccgacgccatcttcaccagcagctacaggaggatcctgggccagctgtacgctaggaagctcctgcacgagatcatgaacaggcagcagggcgagaggaaccaggagcagaggagcaggttcaactgataagcttatcggggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggctcgagggggggcccggtaccagcttttgttccctttagtgagggttaatttcgagcttggtcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggctagcgcttagaagaactcatccagcagacggtagaatgcaatacgttgagagtctggagctgcaataccatacagaaccaggaaacggtcagcccattcaccacccagttcctctgcaatgtcacgggtagccagtgcaatgtcctggtaacggtctgcaacacccagacgaccacagtcaatgaaaccagagaaacgaccattctcaaccatgatgttcggcaggcatgcatcaccatgagtaactaccaggtcctcaccatccggcatacgagctttcagacgtgcaaacagttcagccggtgccagaccctgatgttcctcatccaggtcatcctggtcaaccagacctgcttccatacgggtacgagcacgttcaatacgatgttttgcctggtggtcaaacggacaggtagctgggtccagggtgtgcagacgacgcattgcatcagccatgatagaaactttctctgccggagccaggtgagaagacagcaggtcctgacccggaacttcacccagcagcagccagtcacgaccagcttcagtaactacatccagaactgcagcacacggaacaccagtggttgccagccaagacagacgagctgcttcatcctgcagttcattcagagcaccagacaggtcagttttaacaaacagaactggacgaccctgtgcagacagacggaaaacagctgcatcagagcaaccaatggtctgctgtgcccagtcataaccaaacagacgttcaacccaggctgccggagaacctgcatgcagaccatcctgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagcaactgttgggaagggcgatcg

SEQ ID NO: 19 - DNA, 2704 bp, artificial sequence
  Plasmid vector with optimized analog bGHRH codon sequence.
tgtaatacgactcactatagggcgaattggagctccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagcggatcccaaggcccaactccccgaaccactcagggtcctgtggacagctcacctagctgccatggtgctgtgggtgttcttcctggtgaccctgaccctgagcagcggctcccacggctccctgccctcccagcctctgcgcatccctcgctacgccgacgccatcttcaccaacagctaccgcaaggtgctcggccagctcagcgcccgcaagctcctgcaggacatcatgaaccggcagcagggcgagcgcaaccaggagcagggagcctgataagcttatcggggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggctcgagggggggcccggtaccagcttttgttccctttagtgagggttaatttcgagcttggtcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggctagcgcttagaagaactcatccagcagacggtagaatgcaatacgttgagagtctggagctgcaataccatacagaaccaggaaacggtcagcccattcaccacccagttcctctgcaatgtcacgggtagccagtgcaatgtcctggtaacggtctgcaacacccagacgaccacagtcaatgaaaccagagaaacgaccattctcaaccatgatgttcggcaggcatgcatcaccatgagtaactaccaggtcctcaccatccggcatacgagctttcagacgtgcaaacagttcagccggtgccagaccctgatgttcctcatccaggtcatcctggtcaaccagacctgcttccatacgggtacgagcacgttcaatacgatgttttgcctggtggtcaaacggacaggtagctgggtccagggtgtgcagacgacgcattgcatcagccatgatagaaactttctctgccggagccaggtgagaagacagcaggtcctgacccggaacttcacccagcagcagccagtcacgaccagcttcagtaactacatccagaactgcagcacacggaacaccagtggttgccagccaagacagacgagctgcttcatcctgcagttcattcagagcaccagacaggtcagttttaacaaacagaactggacgaccctgtgcagacagacggaaaacagctgcatcagagcaaccaatggtctgctgtgcccagtcataaccaaacagacgttcaacccaggctgccggagaacctgcatgcagaccatcctgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagcaactgttgggaagggcgatcg

SEQ ID NO: 20 - DNA, 2704 bp, artificial sequence
  Plasmid vector with optimized analog oGHRH codon sequence.
tgtaatacgactcactatagggcgaattggagctccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagcggatcccaaggcccaactccccgaaccactcagggtcctgtggacagctcacctagctgccatggtgctgtgggtgttcttcctggtgaccctgaccctgagcagcggaagccacggcagcctgcccagccagcccctgaggatccctaggtacgccgacgccatcttcaccaacagctacaggaagatcctgggccagctgagcgctaggaagctcctgcaggacatcatgaacaggcagcagggcgagaggaaccaggagcagggcgcctgataagcttatcggggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggctcgagggggggcccggtaccagcttttgttccctttagtgagggttaatttcgagcttggtcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggctagcgcttagaagaactcatccagcagacggtagaatgcaatacgttgagagtctggagctgcaataccatacagaaccaggaaacggtcagcccattcaccacccagttcctctgcaatgtcacgggtagccagtgcaatgtcctggtaacggtctgcaacacccagacgaccacagtcaatgaaaccagagaaacgaccattctcaaccatgatgttcggcaggcatgcatcaccatgagtaactaccaggtcctcaccatccggcatacgagctttcagacgtgcaaacagttcagccggtgccagaccctgatgttcctcatccaggtcatcctggtcaaccagacctgcttccatacgggtacgagcacgttcaatacgatgttttgcctggtggtcaaacggacaggtagctgggtccagggtgtgcagacgacgcattgcatcagccatgatagaaactttctctgccggagccaggtgagaagacagcaggtcctgacccggaacttcacccagcagcagccagtcacgaccagcttcagtaactacatccagaactgcagcacacggaacaccagtggttgccagccaagacagacgagctgcttcatcctgcagttcattcagagcaccagacaggtcagttttaacaaacagaactggacgaccctgtgcagacagacggaaaacagctgcatcagagcaaccaatggtctgctgtgcccagtcataaccaaacagacgttcaacccaggctgccggagaacctgcatgcagaccatcctgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagcaactgttgggaagggcgatcg

SEQ ID NO: 21 - DNA, 2713 bp, artificial sequence
  Plasmid vector with optimized analog cGHRH codon sequence.
tgtaatacgactcactatagggcgaattggagctccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagcggatcccaaggcccaactccccgaaccactcagggtcctgtggacagctcacctagctgccatggccctgtgggtgttctttgtgctgctgaccctgacctccggaagccactgcagcctgccacccagcccacccttccgcgtcaggcgccacgccgacggcatcttcagcaaggcctaccgcaagctcctgggccagctgagcgcacgcaactacctgcacagcctgatggccaagcgcgtgggcagcggactgggagacgaggccgagcccctgagctgataagcttatcggggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggctcgagggggggcccggtaccagcttttgttccctttagtgagggttaatttcgagcttggtcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggctagcgcttagaagaactcatccagcagacggtagaatgcaatacgttgagagtctggagctgcaataccatacagaaccaggaaacggtcagcccattcaccacccagttcctctgcaatgtcacgggtagccagtgcaatgtcctggtaacggtctgcaacacccagacgaccacagtcaatgaaaccagagaaacgaccattctcaaccatgatgttcggcaggcatgcatcaccatgagtaactaccaggtcctcaccatccggcatacgagctttcagacgtgcaaacagttcagccggtgccagaccctgatgttcctcatccaggtcatcctggtcaaccagacctgcttccatacgggtacgagcacgttcaatacgatgttttgcctggtggtcaaacggacaggtagctgggtccagggtgtgcagacgacgcattgcatcagccatgatagaaactttctctgccggagccaggtgagaagacagcaggtcctgacccggaacttcacccagcagcagccagtcacgaccagcttcagtaactacatccagaactgcagcacacggaacaccagtggttgccagccaagacagacgagctgcttcatcctgcagttcattcagagcaccagacaggtcagttttaacaaacagaactggacgaccctgtgcagacagacggaaaacagctgcatcagagcaaccaatggtctgctgtgcccagtcataaccaaacagacgttcaacccaggctgccggagaacctgcatgcagaccatcctgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagcaactgttgggaagggcgatcg

SEQ ID NO: 22 - DNA, 55 bp, artificial sequence
  Sequence of a human growth hormone (“hGH”) 5′ UTR.
caaggcccaactccccgaaccactcagggtcctgtggacagctcacctagctgcc

SEQ ID NO: 23 - DNA, 782 bp, artificial sequence
  This is a nucleic acid sequence of a plasmid pUC-18 origin of replicaiton
tcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatccttt

SEQ ID NO: 24 - DNA, 5 bp, artificial sequence
  This is a NEO ribosomal binding site.
tcctc

SEQ ID NO: 25 - DNA, 29 bp, artificial sequence
  This is a nucleic acid sequence of a prokaryotic PNEO promoter.
accttaccagagggcgccccagctggcaa

(19) United States
(12) Patent Application Publication
(10) Pub. No.: US 2004/0014645 A1
(43) Pub. Date: Jan. 22, 2004

(54) Increased delivery of a nucleic acid construct in vivo by the poly-L-glutamate ("PLG") system

(75) Inventors: Ruxandra Draghia-Akli, Houston, TX (US); Robert H. Carpenter, Bastrop, TX (US); Douglas R. Kern, The Woodlands, TX (US); Leigh Anne Hill, Houston, TX (US); Heather Attra, Houston, TX (US); Henry Hebel, The Woodlands, TX (US)

Correspondence Address: T. Ling Chwang, Suite 600, 2435 N. Central Expressway, Richardson, TX, 75080, US

(73) Assignee: ADVISYS, Inc., The Woodlands, TX

(21) Appl. No.: 10/395,709

(22) Filed: Mar. 24, 2003

Related U.S. Application Data
(63) Continuation-in-part of application No. 10/156,670, filed on May 28, 2002.

(51) Int. Cl. A61K 48/00; C12N 15/85

(52) U.S. Cl. 514/8; 514/44; 435/455; 435/320.1

(57) ABSTRACT

Plasmid DNA delivered by injection/electroporation to the skeletal muscle can be expressed, and physiologic levels of transgene could be achieved into the circulation. Nevertheless, stabilization of naked DNA may be required and necessary in some cases, as prolonged storage at different temperatures before usage, injection into a large number of animals, etc. It is imperative that the associated compound should not be toxic to the cells (e.g. muscle cells) or cause breakage of plasmid DNA. It would be preferable for the coated DNA to have a similar or increased uptake into the target cells. Low molecular weight poly-L-glutamate compounds have all the desired properties. It was determined that mole/mole ratio DNA/PLG is the optimum concentration for gene therapeutic applications to the skeletal muscle, resulting in increased expression of the transgene, with no damage to the target tissue. Furthermore, stabilization of plasmid DNA by PLG has never been observed or described in the literature.

### RELATED APPLICATIONS

This application is a continuation-in-part of the U.S. patent application Ser. No. 10/156,670 entitled “PLASMID MEDIATED GENE SUPPLEMENTATION AND IN VIVO EXPRESSION OF THE POLY-L-GLUTAMATE (“PLG”) SYSTEM,” and filed on May 25, 2002 with Draghia-Akli et al., listed as inventors, the entirety of the application which is hereby specifically incorporated by reference.

### BACKGROUND

The delivery of isolated or recombinant proteins has been used for many years to correct an array of inborn or acquired deficiencies and imbalances in subjects (e.g. insulin for diabetes). More recently, a nucleic acid expression construct having a specific encoded gene (i.e. a plasmid) was delivered to a somatic tissue and had been shown to be useful for the correction of genetic deficiencies. Although both methods of protein supplementation work well, there are a number of advantages to the nucleic acid expression construct supplementation method when compared to the administration of recombinant proteins, for example: the conservation of native protein structure; improved biological activity; avoidance of systemic toxicities; and avoidance of infectious and toxic impurities. Additionally, the plasmid mediated gene supplementation method allows the subject to have prolonged exposure to a therapeutic range of the therapeutic protein, as demonstrated by the persistent levels of the therapeutic protein found in the subjects circulation system.

The primary limitation of using recombinant protein is the restricted bio-availability of the recombinant protein after each administration. In contrast, bio-availability of plasmid mediated gene supplementation is not an issue because a single plasmid injection into the subject's skeletal muscle permits physiologic expression for extensive periods of time, as disclosed in WO 99/05300 and WO 01/06988. Plasmid DNA constructs are attractive candidate for direct supplementation therapy into the subjects skeletal muscle because plasmid DNA's are well-defined entities, that are biochemically stable and have been used successfully for many years. The relatively low expression levels, achieved after simple plasmid DNA injection are sometimes sufficient to prove bio-activity of secreted peptides (Tsurumi et al., 1996). Although not wanting to be bound by theory, injections of the plasmid constructs can promote the production of enzymes and hormones in subjects in a manner that more closely mimics the natural process. Furthermore, among the non-viral techniques for gene product supplementation in vivo, the direct injection of plasmid DNA into muscle tissue is simple, inexpensive, and safe.

In contrast to viral vectors, a plasmid based expression system can be composed of a synthetic gene delivery system in addition to the nucleic acid encoding a therapeutic gene products. In this way many of the risks associated with viral vectors can be avoided. The plasmid (i.e. a non-viral expression system) products generally have low toxicity due to the use of “species-specific” components for gene delivery, which minimizes the risks of immunogenicity generally associated with viral vectors. To date there have been no reported cases of plasmid vectors becoming integrated into a host chromosomes (Ledwith et al., 2000), which minimizes the risk of adverse effects such as the activation of oncogenes, or the inactivation of tumor suppressor genes during treatment. As episomal systems residing outside the chromosomes, plasmids have defined pharmacokinetics and elimination profiles, leading to a finite duration of gene expression in target tissues (Houk et al., 2001; Mahato et al., 1997).

Unfortunately, most applications for plasmid mediated gene supplementation have suffered from low levels of transgene expression that have resulted from the inefficient uptake of plasmid DNA into the treated tissue cells (Wells et al., 1997). Consequently, the use of plasmid DNA directly injected into a subject for therapy has been limited in the past. For example, the inefficient DNA uptake into muscle fibers after simple direct injection had led to relatively low expression levels, in normal, non-regenerating (Vitadello et al., 1994) or ischemic muscles (Takeshita et al., 1996). Additionally, the duration of the transgene expression has been short (Hartikka et al., 1996) (Danko and Wolff, 1994). Until recently, the most successful previous clinical applications have been confined to vaccines (Davis et al., 1994; Davis et al., 1993).

Thus, extensive efforts have been made to over the past two decades to enhance the delivery of plasmid DNA to cells by both chemical and physical means (Danko et al., 1994). For example, chemical means such as lipofectin/liposome fusion; polylysine condensation with and without adenovirus enhancement have been used with marginal success (Fisher and Wilson, 1994). The use of specific compositions consisting of polyacrylic acid has been disclosed in the International patent publication WO 94/24983. Naked DNA has been administered as disclosed in International patent publication WO/11092. Additionally, physical means of plasmid delivery including electroporation, sonoporation, and pressure. Although each of these methods has had limited success, of all the methods listed, electroporation has been the most promising.

Although not wanting to be bound by theory, the delivery of plasmid DNA into a cell by electroporation involves the application of a pulsed voltage electric field to create transient pores in the cellular membrane that allows for the influx of exogenous plasmid DNA molecules (Smith and Nordstrom, 2000). By adjusting the electrical pulse generated by an electroporetic system, the efficiency of nucleic acid molecules that travel through passageways or pores can be regulated. U.S. Pat. No. 5,704,908 describes an electroporation apparatus for delivering molecules to cells at a selected location within a cavity in the body of a patient. These pulse voltage injection devices are also described in U.S. Pat. Nos. 5,439,440 and 5,702,304, and PCT WO 96/12520,96/12006,95/19805, and 97/07826.

The electroporation technique has been used previously to transfect tumor cells after injection of plasmid DNA (Nishi et al., 1997; Rols et al., 1998), or to deliver the antitumoral drug bleomycin to cutaneous and subcutaneous tumors (Belehradek et al., 1994; Heller et al., 1996). Electroporation also has been used in rodents and other small animals, e.g. (Muramatsu et al., 1998; Aihara and Miyazaki, 1998; Hasegawa et al., 1998; Rizzuto et al., 1999). Advanced techniques of intramuscular injections of plasmid DNA followed by electroporation into skeletal muscle have been shown to lead to high levels of circulating growth hormone releasing hormone (“GHRH”) (Draghia-Akli et al., 1999) (Draghia-Akli et al., 2002b). The in vivo electroporation of the skeletal muscle allows the plasmid DNA to be efficiently taken up in normal fibers, and consequently expressed. Electroporation is the use of an electric field to induce transient permeabilization of bio-membrane pores, and allows macromolecules, ions, and water to pass from one side of the membrane to the other. Thus, electroporation has been used to introduce drugs, DNA or other molecules into multi-cellular tissues. The technique has been used in vivo initially to transfect tumor cells after injection of plasmid DNA (Rols et al., 1998), or to deliver the antitumoral drug bleomycin to cutaneous and subcutaneous tumors (Allegretti and Panje, 2001; Heller et al., 1996). Recently, numerous studies, mostly on small mammals, showed that the technique increases dramatically plasmid uptake by skeletal muscle cells, and allows production of peptides at therapeutic levels (Yasui et al., 2001; Yin and Tang, 2001). Previously, we reported that human growth hormone releasing hormone (“GHRH”) cDNA can be delivered into skeletal muscle by an injectable myogenic expression vector in mice and pigs, where it stimulated growth hormone (“GH”) secretion over a period of at least two months (Draghia-Akli et al., 1997; Draghia-Akli et al., 1999).

Despite the recent advances in the technology of plasmid DNA transfer, additional improvements in electroporation techniques and plasmid DNA compositions are needed. For example, in theory, the entire electroporation procedure can be completed without causing permanent damage to the cell. However, in practice, the electroporation procedure impinges a fatal stress on most cells and leads to degradation of the plasmid DNA (Hartikka et al., 2001). Furthermore, until now, plasmids have been preserved at low temperature prior to usage due to decreased stability and degradation (Evans et al., 2000).

We have now optimized a constant current electroporation delivery technique and a plasmid DNA composition that prevents excessive cellular damage and degradation of the plasmid DNA during the electroporation delivery into muscle cells. For example, during the electroporation process, a transfection facilitation polypeptide (e.g. poly-L-glutamate (“PLG”)) enhances the uptake process. Although not wanting to be bound by theory, several mechanisms for increased uptake may be utilized. For example, the transfection facilitating polypeptide may bind to surface of proteins and facilitate the uptake by increasing the bio-availability, neutralizing the normal degradation process in the interstitial fluid (i.e. protecting the DNA from the nucleases present in the interstitial fluid). In the cells, a transfection facilitating polypeptide may prevent transport of DNA into the lysosomes (i.e. organelles where foreign DNA and/or proteins are degraded in the cells) by disruption of microtubule assembly (Fujii et al., 1986). Although not wanting to be bound by theory, transfection facilitating polypeptides (e.g. PLG groups) naturally occur as attachments to side chains in proteins. Accordingly transfection facilitating polypeptides have been used to increase stability of anti-cancer drugs (Li et al., 2000), and as “glue” to close wounds or to prevent bleeding from tissues during wound and tissue repair (Otani et al., 1998; Otani et al., 1996). Some transfection facilitating polypeptides (e.g. PLG) do not enhance an immune response or the production of antibodies. It should be emphasized that some evidence suggests that certain transfection facilitating polypeptides may only effective in conjunction with the method of electroporation. Furthermore, PLG has been demonstrated to decrease muscle damage associated to plasmid delivery (Draghia-Akli et al., 2002a).

This efficient strategy of utilizing transfection facilitation polypeptides and electroporation for enhancing the electrophoretic delivery of a plasmid DNA construct has been described herein and demonstrated in the skeletal muscle of three different mammalian species. Plasmid stability at high temperatures has been demonstrated.

### SUMMARY

One aspect of the current invention is a composition for facilitating the electrophoretic transfer of a nucleic acid expression construct into the cells of a recipient, wherein the nucleic acid expression construct can express an encoded gene in the recipient. The composition of the invention comprises a nucleic acid expression construct that is associated with a charged transfection-facilitating polypeptide. The composition is prepared wherein a ratio in moles of the charged transfection-facilitating polypeptide to nucleic acid expression construct comprises from 1 mole to 5,000 moles of the charged transfection-facilitating polypeptide per mole of nucleic acid expression construct. In a preferred embodiment, the ratio in moles is equal to 1 mole of the nucleic acid expression construct to 1,200 moles or less of the charged transfection-facilitating polypeptide, and in another preferred embodiment, the ratio in moles is equal to 1 mole of the nucleic acid expression construct to 1 mole of the charged transfection-facilitating polypeptide. In a preferred embodiment the transfection-facilitating polypeptide comprises a charged polypeptide (e.g. poly-L-glutamate). Furthermore, the nucleic acid expression construct comprises SeqID#11, SeqID#12, SeqID#13, SeqID#14, SeqID#17, SeqID#18, SeqID#19, SeqID#20, or SeqID#21. Additionally, the nucleic acid expression construct encodes a growth-hormone-releasing-hormone (“GHRH”) or functional biological equivalent thereof, as embodied in HV-GHRH (SEQID#1), TI-GHRH (SEQID#2), TV-GHRH (SEQID#3),15/27/28-GHRH (SEQID#4), wt-GHRH (SEQID#5).

A second aspect of the current invention is a method for introducing a nucleic acid expression construct into a cell of a selected tissue in a recipient. The method comprises penetrating the selected tissue with a plurality of needle electrodes, wherein the plurality of needle electrodes are arranged in a spaced relationship, introducing a composition comprising nucleic acid expression construct having an associated charged transfection-facilitation polypeptide, and applying an electrical pulse to the plurality of needle electrodes. However, caliper electrodes can also be used an alternative to needle electrodes. The composition is prepared wherein a ratio in moles of the charged transfection-facilitating polypeptide to nucleic acid expression construct comprises from 1 mole to 5,000 moles of the charged transfection-facilitating polypeptide per mole of nucleic acid expression construct. In a preferred embodiment, the ratio in moles is equal to 1 mole of the nucleic acid expression construct to 1,200 moles or less of the charged transfection-facilitating polypeptide, and in another preferred embodiment, the ratio in moles is equal to 1 mole of the nucleic acid expression construct to 1 mole of the charged transfection-facilitating polypeptide. In a preferred embodiment the transfection-facilitating polypeptide comprises a charged polypeptide (e.g. poly-L-glutamate). Furthermore, the nucleic acid expression construct comprises SeqID#11, SeqID#12, SeqID#13, SeqID#14, SeqID#17, SeqID#18, SeqID#19, SeqID#20, or SeqID#21. Additionally, the nucleic acid expression construct encodes a growth-hormone-releasing-hormone (“GHRH”) or functional biological equivalent thereof, as embodied in HV-GHRH (SEQID#1), TI-GHRH (SEQID#2), TV-GHRH (SEQID#3), 15/27/28-GHRH (SEQID#4), wt-GHRH (SEQID#5).

A third aspect of the current invention is a method to increase stability of a nucleic acid expression construct, comprising: mixing the nucleic acid expression construct with a charged transfection-facilitating polypeptide, wherein charged transfection-facilitating polypeptide comprises a poly-L-glutamate polypeptide and the nucleic acid expression construct is utilized for plasmid mediated gene supplementation. The method involves making a composition that is prepared wherein a ratio in moles of the charged transfection-facilitating polypeptide to nucleic acid expression construct comprises from 1 mole to 5,000 moles of the charged transfection-facilitating polypeptide per mole of nucleic acid expression construct. In a preferred embodiment, the ratio in moles is equal to 1 mole of the nucleic acid expression construct to 1,200 moles or less of the charged transfection-facilitating polypeptide, and in another preferred embodiment, the ratio in moles is equal to 1 mole of the nucleic acid expression construct to 1 mole of the charged transfection-facilitating polypeptide. In a preferred embodiment the transfection-facilitating polypeptide comprises a charged polypeptide (e.g. poly-L-glutamate). Furthermore, the nucleic acid expression construct comprises SeqID#11, SeqID#12, SeqID#13, SeqID#14, SeqID#17, SeqID#18, SeqID#19, SeqID#20, or SeqID#21. Additionally, the nucleic acid expression construct encodes a growth-hormone-releasing-hormone (“GHRH”) or functional biological equivalent thereof, as embodied in HV-GHRH (SEQID#1), TI-GHRH (SEQID#2), TV-GHRH (SEQID#3), 15/27/28-GHRH (SEQID#4), wt-GHRH (SEQID#5).

### BRIEF DESCRIPTION OF DRAWINGS

FIG. 1 shows an electrode array of the prior art using six electrodes in three opposed pairs. It further depicts a single centralized electroporation overlap point, which is the center point of the asterisk pattern illustrated;

FIG. 2 shows one electrode array of the present invention using five electrodes. It is further depicts how a symmetrically arranged needle electrode array without opposing pairs can produce a decentralized pattern during an electroporation event in an area where no congruent electroporation overlap points develop and how an area of the decentralized pattern resembles a pentagon;

FIG. 3 shows a the serum levels of SEAP in mice that were injected with an expression plasmid pSP-SEAP coated with various concentrations of poly-L-glutamate;

FIG. 4 shows a the serum levels of SEAP in pigs that were injected with an expression plasmid pSP-SEAP coated with and without poly-L-glutamate.

FIG. 5 shows a the serum levels of SEAP in dogs that were injected with an expression plasmid pSP-SEAP coated with and without poly-L-glutamate.

FIG. 6 shows in vitro increased plasmid DNA stability when poly-L-glutamate is added to the solution. All samples were incubated for 6 month at 37° C.

### DESCRIPTION OF PREFERRED EMBODIMENTS

The term “nucleic acid expression construct” as used herein refers to any type of genetic construct comprising a nucleic acid coding for a RNA capable of being transcribed. The term “expression vector” can also be used interchangeably.

The term “functional biological equivalent” of GHRH as used herein is a polpeptide that has been engineered to contain a distinct amino acid sequence while simultaneously having similar or improved biologically activity when compared to the GHRH

The term “encoded GHRH” as used herein is a biologically active

The term “delivery” as used herein is defined as a means of introducing a material into a subject, a cell or any recipient, by means of chemical or biological process, injection, mixing, electroporation, sonoporation, or combination thereof, either under or without pressure.

The term “subject” as used herein refers to any species of the animal kingdom. In preferred embodiments it refers more specifically to humans and animals used for: pets(e.g. cats, dogs, etc.); work (e.g. horses, cows, etc.); food (chicken, fish, lambs, pigs, etc); and all others known in the art.

The term “recipient” as used herein refers to any species of the animal kingdom. In preferred embodiments it refers more specifically to humans and animals used for: pets(e.g. cats, dogs, etc.); work (e.g. horses, cows, etc.); food (chicken, fish, lambs, pigs, etc); and all others known in the art.

The term “promoter” as used herein refers to a sequence of DNA that directs the transcription of a gene. A promoter may be “inducible”, initiating transcription in response to an inducing agent or, in contrast, a promoter may be “constitutive”, whereby an inducing agent does not regulate the rate of transcription. A promoter may be regulated in a tissue-specific or tissue-preferred manner, such that it is only active in transcribing the operable linked coding region in a specific tissue type or types.

The term “coding region” as used herein refers to any portion of the DNA sequence that is transcribed into messenger RNA (“mRNA”) and then translated into a sequence of amino acids characteristic of a specific polypeptide.

The term “analog” as used herein includes any mutant of GHRH, or synthetic or naturally occurring peptide fragments of GHRH, as HV-GHRH (SEQID#1), TI-GHRH (SEQID#2), TV-GHRH (SEQID#3), 15/27/28-GHRH (SEQID#4), (1-44)NH₂ or (1-40)OH (SEQID#6) forms, or shorter forms to up to (1-29)NH₂.

The term “growth hormone” (“GH”) as used herein is defined as a hormone that relates to growth and acts as a chemical messenger to exert its action on a target cell.

The term “growth hormone releasing hormone” (“GHRH”) as used herein is defined as a hormone that facilitates or stimulates release of growth hormone, and in a lesser extent other pituitary hormones, as prolactin.

The term “molecular switch” as used herein refers molecule that is delivered into a subject that can regulate transcription of a gene.

The term “cassette” as used herein is defined as one or more transgene expression vectors.

The term “post-injection” as used herein refers to a time period following the introduction of a nucleic acid cassette that contains heterologous nucleic acid sequence encoding GHRH or biological equivalent thereof into the cells of the subject and allowing expression of the encoded gene to occur while the modified cells are within the living organism.

The term “placing” as used herein refers to the positioning of a plurality of electrodes (either plate or needle) in a selected tissue.

The term “heterologous nucleic acid sequence” as used herein is defined as a DNA sequence consisting of differing regulatory and expression elements.

The term “vector” as used herein refers to any vehicle that delivers a nucleic acid into a cell or organism. Examples include plasmid vectors, viral vectors, liposomes, or cationic lipids.

The term “electroporation” as used herein refers to a method that utilized electric pulses to deliver a nucleic acid sequence into cells.

The term “electrical pulse” as used herein refers either a constant current pulse, or a constant-voltage pulse.

The term “poly-L-glutamate (“PLG”)” as used herein refers to a biodegradable polymer of L-glutamic acid, in some aspects of the current invention the sodium salt of the said acid is suitable for use as a vector or adjuvant for DNA transfer into cells with or without electroporation.

The term “spaced relationship” as used herein refers to a positioning of electrodes in a tissue of a subject in either a symmetrical or non-symmetrical relationship to other electrodes.

The term “weight ratio” as used herein refers to an amount of nucleic acid expression construct (in micrograms), to an amount of charged transfection-facilitating polypeptide (in micrograms) in a composition, regardless of the total volume delivered.

The term “mole ratio” as used herein refers to an amount of nucleic acid expression construct (in moles), to an amount of charged transfection-facilitating polypeptide (in moles) in a composition.

The standard one and three letter abbreviations for amino acids used herein are as follows: Alanine, A, ala; Arginine, R, arg; Asparagine, N, asn; Aspartic acid, N, asp; Cysteine, C, cys; Glutamine, Q, gln; Glutamic acid, E, glu; Glycine, G, gly; Histidine, H, his; Isoleucine, I, ile; Leucine, L, leu; Lysine, K, lys; Methionine, M, met; Phenylalanine, F, phe; Proline, P, pro; Serine, S, ser; Threonine, T, thr; Tryptophan, W, trp; Tyrosine, Y, tyr; Valine, Y, Val.

The ability of electroporation to enhance plasmid uptake into the skeletal muscle has been well documented. However, effective compositions of nucleic acid expression vectors and transfection facilitating agents for use in electroporation protocols has not been described in the literature. This invention features compositions and methods for enhancing the delivery of a nucleic acid expression construct in a recipient.

Composition formulations: The ability of electroporation to enhance plasmid uptake into the skeletal muscle has been well documented, as described above. Other methods that do not involve electroporation also have been shown to enhance plasmid uptake, for example, a plasmid formulated with transfection facilitating particles poly-L-glutamate (“PLG”) or polyvinylpyrolidone (“PVP”) has been observed to increase gene transfection and consequently increase gene expression to up to 10 fold into mice, rats and dog muscle. One aspect of the current invention is the combination of electroporation and transfection facilitating particles associated with nucleic acid expression constructs. Although not wanting to be bound by theory, PLG will increase the transfection of the plasmid during the electroporation process, not only by physically stabilizing the plasmid DNA, and facilitating the intracellular transport through the membrane pores, but also through an active transporting mechanism. For example, positively charged surface proteins on the cells attract and complex the negatively charged PLG linked to plasmid DNA through protein-protein interactions. When an electric field is applied, the surface proteins reverse direction and actively internalize the DNA molecules. Additionally, PLG/DNA molecules that are in contact with the surface of the cell need only to migrate through the plasma membrane, as opposed to DNA molecules located away from the cell surface in the intercellular space. Thus, protein-protein interactions and proximity of transfection particles may substantially increases the transfection efficiency.

Poly-L-glutamate (“PLG”) is a stable compound, and resistant to high, denaturizing temperatures. PLG has been used previously to increase stability in vaccine preparations because it does not increase the vaccine's immunogenicity. Additionally, PLG has been used as an anti-toxin for post antigen inhalation or exposure top ozone. Plasmid DNA delivered by injection, electroporation, or both to the skeletal muscle are easily expressed, and can be measured as indicated by the physiologic levels of the transgene product in the circulation. Nevertheless, stabilization of naked DNA may be required and is necessary in some cases, as prolonged storage before usage, injection into a large number of animals. As plasmid DNA may be stored in different temperature for variable periods of time, it is critical that plasmid solutions be stable for extended periods of time. It is important that the compound associated with the DNA is not toxic to the cells (e.g. muscle cells) and does not cause breakage of plasmid DNA. It would be preferable for the composition of plasmid DNA and associated transfection facilitating particles to have a similar or increased uptake into the target cells. This invention utilizes low concentrations (e.g. below 6 μg/μl, preferably about 0.01 μg/μl) of low and medium molecular weight poly-L-glutamate (e.g. 1-15 kDa, with an average of 10 kDa or 15-50 kDa, with an average of 35 kDa) compounds display all the desired properties for an effective composition of nucleic acid expression vector and transfection facilitating polypeptide. Although PLG can be used at a high concentration in non-electroporation applications, we have determined that low mole ratio of nucleic acid expression vector to PLG is optimum for electroporation applications to the skeletal muscle. An example of a useful mole ratio of nucleic acid expression vector to PLG is about 1:5,000. Another example of a more useful mole ratio of nucleic acid expression vector to PLG comprises one about 1:2,500. An example of a preferred mole ratio of nucleic acid expression vector to PLG is about 1:1,200. An illustrative mole ratio of nucleic acid expression vector to PLG comprises one about 1:800. A representative mole ratio of nucleic acid expression vector to PLG comprises one about 1:500. An example of a select mole ratio of nucleic acid expression vector to PLG comprises one about 1:200. Another example of an even more select mole ratio of nucleic acid expression vector to PLG comprises one about 1:100. An example of a preferential mole ratio of nucleic acid expression vector to PLG comprises one about 1:50. Another example of a more preferential mole ratio of nucleic acid expression vector to PLG comprises one about 1:20. An example of a even more preferential mole ratio of nucleic acid expression vector to PLG comprises one about 1:10. An example of a most preferred mole ratio of nucleic acid expression vector to PLG is about 1:1.

The proper mole ratio can be calculated for the moles of an appropriately average length nucleic acid expression vector (e.g. in the range of 2,000 bp to 30,000 bp) to moles of PLG of low and medium molecular weight poly-L-glutamate (e.g.1-15 kDa, with an average of 10 kDa or 15-50 kDa, with an average of 35 kDa). The resulting electroporation of a plasmid DNA associated with PLG composition resulted in an increased expression of a reporter transgene and no damage to the target tissue.

Accordingly, the pharmaceutical composition of the present invention may be delivered via various routes and to various sites in an animal body to achieve a particular effect. One skilled in the art will recognize that although more than one route can be used for administration, a particular route can provide a more immediate and more effective reaction than another route. Although not wanting to be bound by theory, local or systemic delivery can be accomplished by administration comprising application or instillation of the formulated composition into body cavities, inhalation or insufflation of an aerosol, or by parenteral introduction, comprising intramuscular, intravenous, peritoneal, subcutaneous, intradermal, as well as topical administration. Additionally, different methods of delivery may be utilized to administer a plasmid/facilitating agent composition into a cell. Examples include: (1) methods utilizing physical means, such as electroporation (electricity), a gene gun (physical force) or applying large volumes of a liquid (pressure); and (2) methods wherein said vector is complexed to another entity, such as a liposome or transporter molecule.

Constant Current Electroporation: The underlying phenomenon of electroporation is believed to be the same in all cases, but the exact mechanism responsible for the observed effects has not been elucidated. Although not wanting to be bound by theory, the overt manifestation of the electroporative effect is that cell membranes become transiently permeable to large molecules, after the cells have been exposed to electric pulses. There are conduits through cell walls, which under normal circumstances, maintain a resting transmembrane potential of ca. 90 mV by allowing bi-directional ionic migration.

Although not wanting to be bound by theory, electroporation makes use of the same structures, by forcing a high ionic flux through these structures and opening or enlarging the conduits. In prior art, metallic electrodes are placed in contact with tissues and predetermined voltages, proportional to the distance between the electrodes are imposed on them. The protocols used for electroporation are defined in terms of the resulting field intensities, according to the formula E=V/d, where (“E”) is the field, (“V”) is the imposed voltage and (“d”) is the distance between the electrodes.

The electric field intensity E has been a very important value in prior art when formulating electroporation protocols for the delivery of a drug or macromolecule into the cell of the subject. Accordingly, it is possible to calculate any electric field intensity for a variety of protocols by applying a pulse of predetermined voltage that is proportional to the distance between electrodes. However, a caveat is that an electric field can be generated in a tissue with insulated electrodes (i.e. flow of ions is not necessary to create an electric field). Although not wanting to be bound by theory, it is the current that is necessary for successful electroporation not electric field per se.

During electroporation, the heat produced is the product of the inter-electrode impedance, the square of the current, and the pulse duration. Heat is produced during electroporation in tissues and can be derived as the product of the inter-electrode current, voltage and pulse duration. The protocols currently described for electroporation are defined in terms of the resulting field intensities E, which are dependent on short voltage pulses of unknown current. Accordingly, the resistance or heat generated in a tissue cannot be determined, which leads to varied success with different pulsed voltage electroporation protocols with predetermined voltages. The ability to limit heating of cells across electrodes can increase the effectiveness of any given electroporation voltage pulsing protocol.

Controlling the current flow between electrodes allows one to determine the relative heating of cells. Thus, it is the current that determines the subsequent effectiveness of any given pulsing protocol, and not the voltage across the electrodes. Predetermined voltages do not produce predetermined currents, and prior art does not provide a means to determine the exact dosage of current, which limits the usefulness of the technique. Thus, controlling an maintaining the current in the tissue between two electrodes under a threshold will allow one to vary the pulse conditions, reduce cell heating, create less cell death, and incorporate macromolecules into cells more efficiently when compared to predetermined voltage pulses.

A constant-current electroporation device is the invention of a co-pending application entitled “Electrode assembly for constant current-electroporation and use” S/N 60/362,362 filed on Mar. 7, 2002 with Westerstein et al., (“the Western '362 application”) listed as inventors, and is herby incorporated by reference. One aspect of the Western '362 application overcomes the above problem by providing a means to effectively control the dosage of electricity delivered to the cells in the inter-electrode space by precisely controlling the ionic flux that impinges on the conduits in the cell membranes. Thus, the precise dosage of electricity to tissues can be calculated as the product of the current level, the pulse length and the number of pulses delivered. The constant-current system, comprises an electrode apparatus connected to a specially designed circuit, which is also utilized in the current invention.

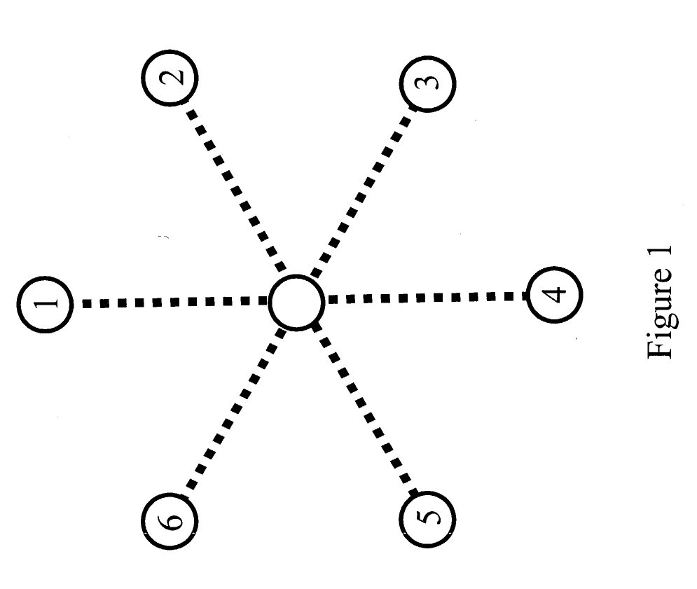

One aspect of the present invention is to provide a means to deliver the electroporative current to a volume of tissue along a plurality of paths without, causing excessive concentration of cumulative current in any one location, thereby avoiding cell death owing to overheating of the tissue. However, the composition of the nucleic acid expression vector associated with a transfection facilitation poly-peptide will further facilitate successful transfection protocols. For example, the maximal energy delivery from a particular pulse would occur along a line that connects two electrodes. Prior art teaches that the electrodes are present in pairs and that the voltage pulses are delivered to the paired electrodes of opposed polarity. Accordingly, the maximal energy delivery from a particular pulse would occur along a line that connects two electrodes. An example of the energy delivery pathway in a prior art electrode, which utilizes three pairs of radial electrodes with a center electrode, is described above and as in FIG. 1. A distribution of the energy crosses at the center point of the electrodes, which may lead to unnecessary heating and decreased survival of cells. Thus, nucleic acid/transfection facilitation composition of the current invention can also help stabilize cells in prior art electroporation protocols.

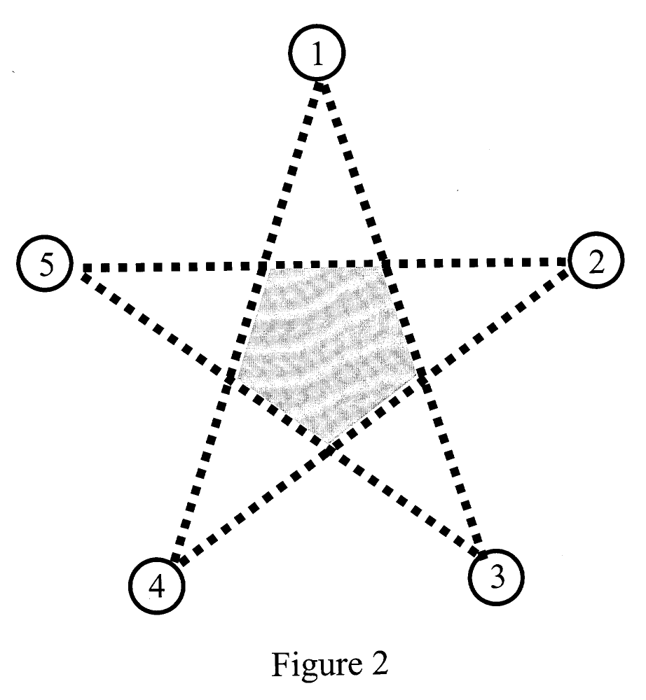

The electrodes of one embodiment of the present invention are arranged in a radial and symmetrical array, but unlike prior art, the electrodes are odd numbered, and not in opposing pairs (FIG. 2). Delivering an electric pulse to any two of the electrodes from an electric pulse generator results in a pattern that is best described as a polygon. Tracing this pattern would result in a five-point star with a pentagon of electrical pulses surrounding the center of the array in tissue where the concentration of molecules to be transfected is greatest. Although not wanting to be bound by theory, it is not the odd number of electrodes, per se, that makes a difference, but in the direction of the current paths. With the configuration of prior art, all the pulses generate a current that passes through the center of the assembly. The cumulated dose, i.e. the heating effect is therefore concentrated in the center, with the peripheral dose falling off rapidly. With the “five-pointed star” arrangement, the dose is spread more evenly, over a larger volume. For example, if the electrodes are arranged in an array of five electrodes, the pulses might be sequentially applied to electrodes 1 and 3, then 3 and 5, then 5 and 2, then 2 and 4, then 4 and 1. However, because the tissue between the electrodes is a volume conductor, a certain current intensity exists along parallel lines, weakening as the distance from the center line increases. The cumulative effect of a sequence of pulses results in a more uniform distribution of the energy delivered to the tissues, increasing the probability that the cells that have been electroporated actually survive the procedure.

It is known in prior art that the nature of the voltage pulse to be generated is determine by the nature of tissue, the size of the selected tissue and distance between electrodes. It is desirable that the voltage pulse be as homogenous as possible and of the correct amplitude. Excessive field strength results in the lysing of cells, whereas a low field strength results in reduced efficacy of electroporation. Prior art inventions utilize the distance between electrodes to calculate the electric field strength and predetermined voltage pulses for electroporation. This reliance on knowing the distance between electrodes is a limitation to the design of electrodes. Because the programmable current pulse controller will determine the impedance in a volume of tissue between two electrodes, the distance between electrodes is not a critical factor for determining the appropriate electrical current pulse. Therefore, an alternative embodiment of the needle electrode array design would be one that is non-symmetrical. In addition, one skilled in the art can imagine any number of suitable symmetrical and non-symmetrical needle electrode arrays that do not deviate from the spirit and scope of a particular electrode design. The depth of each individual electrode within an array and in the desired tissue could be varied with comparable results. In addition, multiple injection sites for the macromolecules could be added to the needle electrode array.

By utilizing the constant current electroporation device described in the Western '362 application a simple means for determining the temperature elevation of the tissues exposed to the pulses is available. For example, the product of the measured inter-electrode impedance, the square of the current and the cumulated pulse duration is a measure of the total energy delivered. This quantity can be converted to degrees Celsius, when the volume of the tissues encompassed by the electrodes and the specific heat of the tissues are known. For example the rise in tissue temperature (“T”, Celsius) is the resistance (“R”, ohms), current (“I”, Amperes), length of pulse (“t”, seconds), and the conversion factor between joules and calories (“K”). T=RI²tK.

At the moment of electroporation, the current increases in a prior art system where a predetermined voltage has been imposed on the electrodes, owing to the fact that increased cell permeability lowers the inter-electrode impedance. This may lead to an excessive temperature rise, resulting in cell death. For example, utilizing values common for conventional electroporators, and assuming that the volume enclosed by the electrodes is one cubic centimeter and the specific heat of the tissues is close to unity, the temperature rise owing to one 50 msec pulse with an average current of 5 Amperes across a typical load impedance of 25 ohms is ca 7.5° C. This points out the necessity of inserting an adequate delay between successive pulses, to allow the subjects circulatory system to remove enough heat so that the cumulative temperature rise will not result in destruction of the tissues being electroporated.

The advantage of a constant-current is that the pulse can be prevented from attaining an amplitude at which the cells are destroyed. In a predetermined voltage system, the current can attain a destructive intensity, and the operator can not prevent that from happening. In a constant-current system, the current is preset under a threshold level where cell death does not occur. The exact setting of the current is dependent of the electrode configuration, and it must be determined experimentally. However, once the proper level has been determined, cell survival is assured, from case to case. The addition of a nucleic acid expression construct associated with a transfection facilitating polypeptide increases the opportunity of electroporated cells to incorporate the plasmid construct.

Nucleic acid constructs for therapy: One aspect of this invention relates to a composition and method for efficient delivery of a nucleic acid construct to a tissue as a treatment for various diseases found in chronically ill subjects. More specifically, the aspects of this invention pertain to a method for delivering a heterologous nucleic acid sequence that is encoding a specific gene (e.g. growth hormone releasing hormone (“GHRH”) or biological equivalent thereof) into one or more cells of the subject (e.g. somatic, stem, or germ cells) and allowing expression of the encoded gene (e.g. GHRH or biological equivalent thereof) to occur while the modified cells are within the subject. The method of delivering the nucleic acid sequence encoding the gene is via electroporation. The subsequent expression of the encoded gene can be regulated by a tissue specific promoter (e.g. muscle), and/or by a regulator protein that contains a modified ligand-binding domain (e.g. molecular switch), which will only be active when the correct modified ligand (e.g. mifepistone) is externally administered into the subject. For example, the extracranial expression and ensuing release of GHRH or biological equivalent thereof by the modified cells can be used to treat anemia, wasting, immune dysfunction, life extension or other disorders in the chronically ill subject.

Recombinant GH replacement therapy is widely used clinically, with beneficial effects, but generally, the doses are supraphysiological. Such elevated doses of recombinant GH are associated with deleterious side-effects, for example, up to 30% of the recombinant GH treated patients report a higher frequency of insulin resistance or accelerated bone epiphysis growth and closure in pediatric patients. In addition, molecular heterogeneity of circulating GH may have important implications in growth and homeostasis, which can lead to a less potent GH that has a reduced ability to stimulate the prolactin receptor. These unwanted side effects result from the fact that treatment with recombinant exogenous GH protein raises basal levels of GH and abolishes the natural episodic pulses of GH. In contradistinction, no side effects have been reported for recombinant GHRH therapies. The normal levels of GHRH in the pituitary portal circulation range from 150-to-800 pg/ml, while systemic circulating values of the hormone are up to 100-500 pg/ml. Some patients with acromegaly caused by extracranial tumors have level that is nearly 100 times as high (e.g. 50 ng/ml of immunoreactive GHRH). Long term studies using recombinant GHRH therapies (1-5 years) in children and elderly humans have shown an absence of the classical GH side-effects, such as changes in fasting glucose concentration or, in pediatric patients, the accelerated bone epiphysal growth and closure or slipping of the capital femoral epiphysis. Thus, recombinant GHRH therapy may be more physiological than recombinant GH therapy. Unfortunately, due to the short half-life of the peptide in vivo, frequent (i.e. one to three times a day) intravenous or subcutaneous administration is necessary if the recombinant protein is used. A gene transfer approach, however could overcome this limitations to GHRH use. Moreover, a wide range of doses can be therapeutic. The choice of GHRH for a gene therapeutic application is favored by the fact that the gene, cDNA and native and several mutated molecules have been characterized for the pig and other species, and the measurement of therapeutic efficacy is straightforward and unequivocal.

The invention may be better understood with reference to the following examples, which are representative of some of the embodiments of the invention, and are not intended to limit the invention.

### EXAMPLE 1

Plasmid vectors containing the muscle specific synthetic promoter SPc5-12 were previously described (Li et al., 1999). Wild type and mutated porcine GHRH cDNAs were generated by site directed mutagenesis of GHRH CDNA (Altered Sites II in vitro Mutagenesis System, Promega, Madison, Wis.), and cloned into the BamHI/Hind III sites of pSPc5-12, to generate pSP-wt-GHRH, or pSP-HV-GHRH respectively. The 3′ untranslated region (3′UTR) of growth hormone was cloned downstream of GHRH cDNA. The resultant plasmids contained mutated coding region for GHRH, and the resultant amino acid sequences were not naturally present in mammals. Although not wanting to be bound by theory, the effects on treating anemia; increasing total red blood cell mass in a subject; reversing the wasting; reversing abnormal weight loss; treating immune dysfunction; reversing the suppression of lymphopoesis; or extending life expectancy for the chronically ill subject are determined ultimately by the circulating levels of analog GHRH hormones. Several different plasmids that encoded different mutated amino acid sequences of GHRH or functional biological equivalent thereof are as follows:

| | |
|---|---|
| Plasmid | Encoded Amino Acid Sequence |

| | | |
|---|---|---|
| wt-GHRH | YADAIFTNSYRKVLGQLSARKLLQDLMSRQQGERNQEQGA-OH | (SEQ ID#5) |
| HV-GHRH | HVDAIFTNSYRKVLAQLSARKLLQDLLNRQQGERNQEQGA-OH | (SEQ ID#1) |
| TI-GHRH | YIDAIFTNSYRKVLAQLSARKLLQDILNRQQGERNQEQGA-OH | (SEQ ID#2) |
| TV-GHRH | YVDAIFTNSYRKVLAQLSARKLLQDILNRQQGERNQEQGA-OH | (SEQ ID#3) |
| 15/27/28-GHRH | YADAIFTNSYRKVLAQLSARKLLQDILNRQQGERNQEQGA-OH | (SEQ ID#4) |

In general, the encoded GHRH or functional biological equivalent thereof is of formula (SeqID#6):

-A₋₁-A₂-DAIFTNSYRKVL-A₃-QLSARKLLQDI-A₄-A₅-RQQGERNQEQGA-OH

wherein: a standard one letter amino acid abbreviation is used; and A₁ is a D-or L-isomer of an amino acid selected from the group consisting of tyrosine (“Y”), or histidine (“H”); A₂ is a D-or L-isomer of an amino acid selected from the group consisting of alanine (“A”), valine (“V”), or isoleucine (“I”); A₃ is a D-or L-isomer of an amino acid selected from the group consisting of alanine (“A”) or glycine (“G”); A₄ is a D-or L-isomer of an amino acid selected from the group consisting of methionein (“M”), or leucine (“L”); A₅ is a D-or L-isomer of an amino acid selected from the group consisting of serine (“S”) or asparagines (“N”).

Another plasmid that was utilized included the pSP-SEAP construct that contains the SacI/HindIII SPc5-12 fragment, SEAP gene and SV40 3′UTR from pSEAP-2 Basic Vector (Clontech Laboratories, Inc., Palo Alto, Calif.).

The plasmids described above do not contain polylinker, IGF-I gene, a skeletal α-actin promoter or a skeletal α-actin 3′ UTR (untranslated region)/NCR (non-coding region). Furthermore, these plasmids were introduced by muscle injection, followed by in vivo electroporation, as described below.

In terms of “functional biological equivalents,” it is well understood by the skilled artisan that, inherent in the definition of a “biologically functional equivalent” protein and/or polynucleotide, is the concept that there is a limit to the number of changes that may be made within a defined portion of the molecule while retaining a molecule with an acceptable level of equivalent biological activity. Functional biological equivalents are thus defined herein as those proteins (and polynucleotides) in selected amino acids (or codons) may be substituted. A peptide comprising a functional biological equivalent of GHRH is a polypeptide that has been engineered to contain distinct amino acid sequences while simultaneously having similar or improved biologically activity when compared to GHRH. For example one biological activity of GHRH is to facilitate growth hormone (“GH”) secretion in the subject.

Plasmid associated with PLG in mice. In order to demonstrate the improved uptake of electroporated cells with a composition of a nucleic acid expression construct associated with a transfection facilitating polypeptide, a series of electroporation experiments were designed. Three separate sets of experiments were conducted in mice. All mice were given a total of 30 μg (micrograms) pSP-SEAP (approximately 5,000 base pairs (“bp”)), +/− PLG (weighted average MW=10,900) in a total volume of 25 μl (microliters). One group of 10 mice received naked, non-coated plasmid; the subsequent groups received plasmid coated with decreasing concentrations of PLG (see Table 1 below):

| | Total Inj. | | | | Approximate |
|---|---|---|---|---|---|
| | Vol | DNA | PLG | Total PLG | Mole ratio |
| Group | (μl) | (μl) | (μg/μl) | (μg) | DNA:PLG |
| 1 | 25 | 30 | 0.00 | 0.00 | — |
| 2 | 25 | 30 | 6.00 | 150 | 1:1200 |
| 3 | 25 | 30 | 1.00 | 25 | 1:200 |
| 4 | 25 | 30 | 0.10 | 2.50 | 1:20 |
| 5 | 25 | 30 | 0.01 | 0.25 | 1:2 |

The mole ratios are provided for the purpose of example. The mole ratio listed in Table 1 are based upon a 5,000 bp nucleic acid expression vector, and PLG with the weighted average molecular weight of 10,900. For example group 2 in Table 1 has an injection total of 30 g of DNA vector associated with 150 μg of transfection facilitating polypeptides, wherein the mole ratio is 1:1,200. Another example of group 3 in Table 1 has an injection total of 30 μg of DNA vector associated with 0.25 μg of transfection facilitating polypeptides, wherein the mole ratio is less than 1:2. Mole ratio's of DNA vector to PLG having a 1:1 relationship comprises a lower limit formulation still having a higher transfection efficiency than a “naked” DNA vector alone. One of ordinary skill in the art is capable of formulating mole ratio calculations with different length expression vectors and variable molecular weights of PLG. Additionally, it is understood that the length of the nucleic acid expression vector and the weighted average molecular weight of PLG is subject to change based upon specific vector lengths and particular formulation strategies known to one skilled in the art (e.g. functional nucleic acid expression vectors greater than or less than about 5,000 nucleotides, and PLG having an average molecular weight of less than about 1 to about 30 kDa). Consequently, even the smallest of PLG polymers (e.g. PLG trimers having a molecular weight˜400 Da) can be used for this invention.

Electroporation was carried out using a constant current electroporation apparatus that is the subject of the Western '362 co-pending application. This device was used to deliver square wave pulses in all experiments. The amplitude conditions of 1 mA, 5 pulses, 50 milliseconds per pulse were used. Caliper electrodes were used to deliver in vivo electric pulses. The caliper (plate) electrodes consisted of 1.5 cm square metallic blocks mounted on a ruler, so the distance between the plates could be easily assessed. Plasmid DNA or associated DNA was injected through the intact skin into the tibialis anterior muscle of mice. Each animal received one injection into a single injection site. Although a constant-current electroporation device was used in specific examples, it is not intended to limit general embodiments of the invention (i.e. other electroporation devices may provide satisfactory results.) Furthermore, the order of the placement of the electrodes and subsequent injection of plasmid are not sequentially limiting.

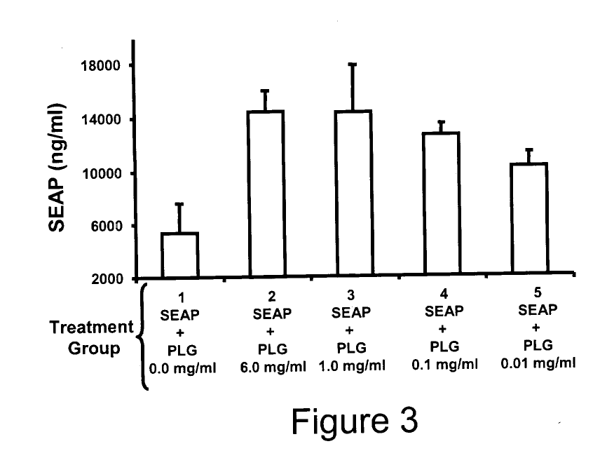

In order to determine the amount of expression of the SEAP gene product that was encoded on the DNA vector, mice were bled and serum collected for up to 3 month post-injection. The SEAP molecule usually disappears after birth, and it is immunogenic in adult animals. Blood was collected by tail vein collection for mice, before plasmid administration, and up to 3 month post-injection in mice. Serum levels of SEAP were determined using a chemiluminescence assay (Tropix, Bedford, Mass.) following the manufacturer instructions. FIG. 3 shows the serum SEAP levels for all five groups of mice described in Table 1. Although naked plasmid (Group 1, FIG. 3) showed some expression, all groups with the nucleic acid expression vector associated with PLG (groups 2-5, FIG. 3) showed significantly higher serum levels of SEAP. Nevertheless, when samples from selected animals from each group were analyzed by histochemistry for inflammation markers (e.g. macrophages, B-cells, and counterstained with hematoxilin/eosin), mice from group 5 (i.e. nucleic acid expression construct coated with 0.01 μg/μl PLG) had the least inflammation associated with the delivery procedure at 3 days post-injection. Despite higher expression at earlier time points, group 2 injected with plasmid associated with 6 μg/μl had high inflammation and some morphological changes. This observation correlates with the data in the literature, that shows short-term enhanced expression using PLG compounds, expression that disappears at approximately 1 month post-injection. (Fewell et al., 2001).

Histological analysis—Muscle and skin samples were fixed overnight, dehydrated in alcohol and paraffin embedded. Five microns sections were cut and stained with hematoxilin/eosin (Sigma Chemical, St. Louis, Mo.). Serial sections were stained with picric acid. Digital images of the slides were captured using a CoolSnap digital color camera (Roper Scientific, Tucson, Ariz.) with MetaMorph software (Universal Imaging Corporation, Downington, Pa.) and a Zeiss Axioplan 2 microscope with a (×40) objective (numerical aperture 0.75 plan).

Statistics—Data are analyzed using STATISTICA analysis package (StatSoft, Inc. Tulsa, Okla.). Values shown in the figures are the mean±s.e.m. Specific P values were obtained by comparison using ANOVA. A P<0.05 was set as the level of statistical significance.

### EXAMPLE 2

### PLG Coating in Pigs

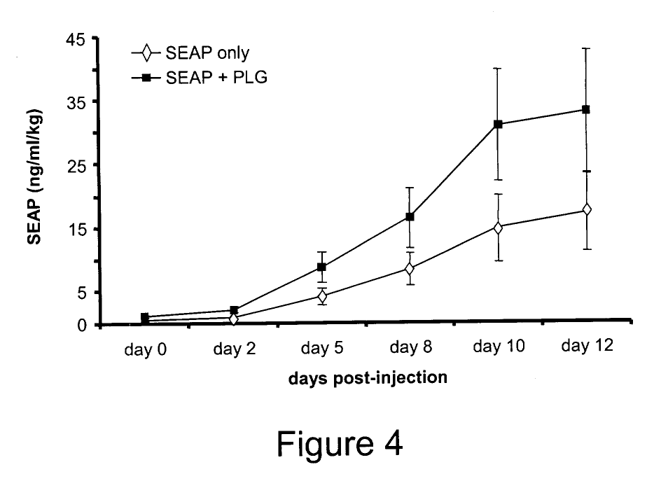

In order to demonstrate similar results in a larger mammal, experiments similar to Example 1 above were conducted in pigs. Thus, two groups of three pigs were injected with 500 μg (micrograms) of pSP-SEAP and electroporated. The plasmid expressed secreted embryonic alkaline phosphatase (“SEAP”). The molecule usually disappears after birth, and it is immunogenic in adult animals. One group received naked nucleic acid construct and the second group received the nucleic acid construct in 0.01 μg/μl PLG Pigs were weighted and bled prior to injection, and every other day up to 10 days post-injection. Serum was collected from pigs by jugular puncture before plasmid injection, and at 2, 4, 6, 8 and 10 days for the SEAP studies. Serum levels of SEAP were determined using a chemiluminescence assay (Tropix, Bedford, Mass.) following the manufacturer instructions. SEAP assay (FIG. 4) showed an increased expression in animals injected with PLG coated plasmid versus naked plasmid throughout 12 days of experiment (32.9±19.3 ng/ml/kg in PLG/plasmid pig versus 17.14±12.44 ng/ml/kg in animals injected with naked plasmid). Although not wanting to be bound by theory, the increased expression may be attributed to the increased stability of plasmid, facilitation of transfection into the muscle cells, or both.

Electroporation devices A constant current electroporator machine (Advisys, Inc.) was used to deliver square wave pulses in all experiments. The electroporation parameters included an amplitude condition of 1 mA, 5 pulses, 50 milliseconds per pulse. A needle electrode was used to deliver in vivo electric pulses. The 5-needle electrode device consists of a circular array (1 cm diameter) of equally spaced filled 21-gauge needles mounted on a non-conductive material. All needles were 2 cm in length and during all injections or electroporations, the needles were completely inserted into the muscle. Plasmid DNA was injected through the intact skin into the semitendinosous muscle of pigs with a 21 g needle. Each animal received one injection into a single injection site and the injection site also received a tattoo so it could be easily isolated at the end of the experiment.

Histological analysis—Muscle and skin samples were fixed overnight, dehydrated in alcohol and paraffin embedded. Five microns sections were cut and stained with hematoxilin/eosin (Sigma). Serial sections were stained with picric acid. Digital images of the slides were captured using a CoolSnap digital color camera (Roper Scientific, Tucson, Ariz.) with MetaMorph software (Universal Imaging Corporation, Downington, Pa.) and a Zeiss Axioplan 2 microscope with a (×40) objective (numerical aperture 0.75 plan).

Statistics—Data are analyzed using STATISTICA analysis package (StatSoft, Inc. Tulsa, Okla.). Values shown in the figures are the mean±s.e.m. Specific P values were obtained by comparison using ANOVA. A P<0.05 was set as the level of statistical significance.

### EXAMPLE 3

### PLG Coating in Dogs

In order to demonstrate similar results in a different species of larger mammal, experiments similar to Example 2 were conducted in dogs. Thus, a comparison of expression in dogs injected with 5 needle array electrodes, with coated or naked plasmid. Four groups of 5 dogs were injected with a plasmid DNA, pSP-SEAP, expressing the secreted embryonic alkaline phosphatase (“SEAP”). The molecule usually disappears after birth, and it is immunogenic in adult animals. No adverse reaction, or change in biochemical, clinical or hormonal profiles is related to the development of the immune response to SEAP in animals. As described above, the injection was followed by electroporation, using standard conditions, and 5 needle electrodes. The plasmid DNA was either naked, or coated with a mol/mol dilution of poly-L-glutamate. The groups are as follows:

Group 1-5 needle (5N), 0.5 mg, naked (NK)

Group 2-5 needle (5N), 0.1 mg, naked (NK)

Group 3-5 needle (5N), 0.5 mg, coated (PLG)

Group 4-5 needle (5N), 0.1 mg, coated (PLG)

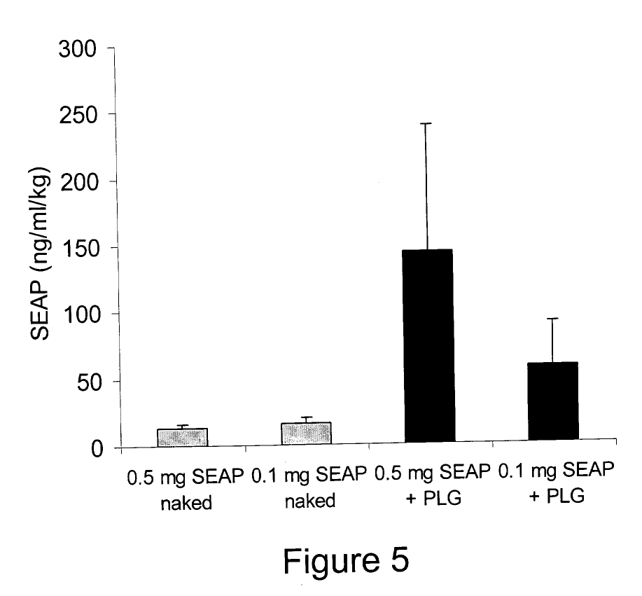

Dogs were weight and bled at baseline (pre-injection) and every other day to day 10 post-injection. Serum was assayed for SEAP. Values were corrected for weight (blood volume). SEAP values were analyzed for differences in between the different injected groups. The results of these experiments are shown in FIG. 5. The results showed that a 5 needle electrode could be used in dogs to efficiently mediate electroporation. Additionally, PLG coated DNA increasing plasmid stability and electroporation efficiency in dogs.

### EXAMPLE 4

### PLG Increases Plasmid Stability in Vitro at High Temperatures

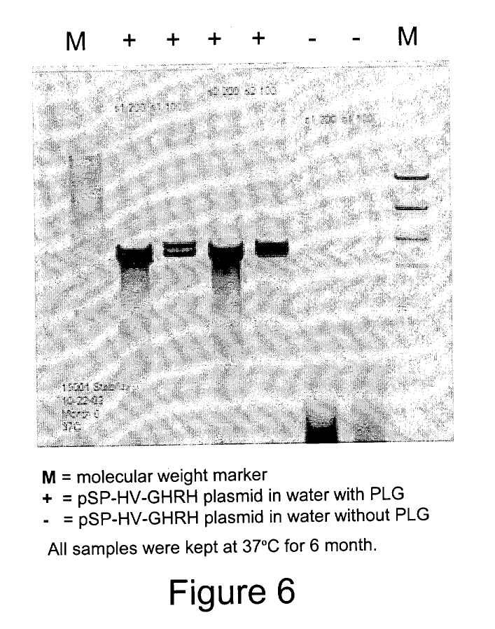

In order to evaluate the effects of the PLG on plasmid stability, the following assay has been performed. Plasmid pSP-HV-GHRH encoding for a super-porcine growth hormone releasing hormone was diluted into distilled water to a final concentration of 2 mg/ml. PLG in a mol/mol ratio was added to a group of samples, while PLG was not added into control samples. All samples were incubated for 6 month at 37° C. After 6 month, aliquots were taken from all samples, and run onto an agarose gel (FIG. 6). As seen in the gel image, all plasmid is present in the samples were PLG was added, while in control samples all plasmid is completely degraded.

One skilled in the art readily appreciates that the patent invention is well adapted to carry out the objectives and obtain the ends and advantages mentioned as well as those inherent therein. Growth hormone, growth hormone releasing hormone, analogs, plasmids, vectors, charged transfection facilitating polypeptides, poly-L-glutamate, pharmaceutical compositions, treatments, electroporation methods, procedures and other techniques described herein are presently representative of several aspects of the current invention and are intended to be exemplary and are not intended as limitations of the scope. Changes therein and other uses will occur to those skilled in the art which are encompassed within the spirit of the invention or defined by the scope of the pending claims.

Accordingly, the present invention provides a method of transferring a therapeutic gene to a host, which comprises administering the vector of the present invention, preferably as part of a composition, using any of the aforementioned routes of administration or alternative routes known to those skilled in the art and appropriate for a particular application. Effective gene transfer of a vector to a host cell in accordance with the present invention to a host cell can be monitored in terms of a therapeutic effect (e.g. alleviation of some symptom associated with the particular disease being treated) or, further, by evidence of the transferred gene or expression of the gene within the host (e.g., using the polymerase chain reaction in conjunction with sequencing, Northern or Southern hybridizations, or transcription assays to detect the nucleic acid in host cells, or using immunoblot analysis, antibody-mediated detection, mRNA or protein half-life studies, or particularized assays to detect protein or polypeptide encoded by the transferred nucleic acid, or impacted in level or function due to such transfer).

These methods described herein are by no means all-inclusive, and further methods to suit the specific application will be apparent to the ordinary skilled artisan. Moreover, the effective amount of the compositions can be further approximated through analogy to compounds known to exert the desired effect.

Furthermore, the actual dose and schedule can vary depending on whether the compositions are administered in combination with other pharmaceutical compositions, or depending on inter-individual differences in pharmacokinetics, drug disposition, and metabolism. Similarly, amounts can vary in in vitro applications depending on the particular cell line utilized (e.g., based on the number of vector receptors present on the cell surface, or the ability of the particular vector employed for gene transfer to replicate in that cell line). Furthermore, the amount of vector to be added per cell will likely vary with the length and stability of the therapeutic gene inserted in the vector, as well as the nature of the sequence, and is particularly a parameter which needs to be determined empirically, and can be altered due to factors not inherent to the methods of the present invention (for instance, the cost associated with synthesis). One skilled in the art can easily make any necessary adjustments in accordance with the exigencies of the particular situation.

What is claimed is:

1. A composition comprising:
(a) a nucleic acid expression construct; and
(b) a charged transfection-facilitating polypeptide associated therewith;
wherein a ratio in moles of the charged transfection-facilitating polypeptide to nucleic acid expression construct comprises from 1 mole to 5,000 moles of the charged transfection-facilitating polypeptide per mole of nucleic acid expression construct.

2. The composition of claim 1, wherein the charged transfection-facilitating polypeptide comprises poly-L-glutamate.

3. The composition of claim 1, wherein the ratio in moles of the charged transfection-facilitating polypeptide to nucleic acid expression construct is equal to 1,200 moles or less of the charged transfection-facilitating polypeptide per mole of nucleic acid expression construct.

4. The composition of claim 1, wherein the ratio in moles of the charged transfection-facilitating polypeptide to nucleic acid expression construct is equal to 1 mole of the charged transfection-facilitating polypeptide per mole of nucleic acid expression construct.

5. The composition of claim 1, wherein an average molecular length of the nucleic acid expression vector is from about 2,000 to about 5,000 nucleotide base pairs.

6. The composition of claim 1, wherein an average molecular weight of the charged transfection-facilitating polypeptide is from about 400 to about 30,000 Da.

7. The composition of claim 1, wherein an average molecular length of the nucleic acid expression vector is about 5,000 nucleotide base pairs, and an average molecular weight of the charged transfection-facilitating polypeptide is about 10,900 Da.

8. The composition of claim 1, wherein the nucleic acid expression construct comprises SeqID#11, SeqID#12, SeqID#13, SeqID#14, SeqID#17, SeqID#18, SeqID#19, ScqID#20, or SeqID#21.

9. The composition of claim 1, wherein the nucleic acid expression construct comprises a gene that encodes a growth-hormone-releasing-hormone (“GHRH”) or functional biological equivalent thereof.

10. The composition of claim 9, wherein the encoded GHRH is a biologically active polypeptide, and the encoded functional biological equivalent of GHRH is a polypeptide that has been engineered to contain a distinct amino acid sequence while simultaneously having similar or improved biologically activity when compared to the GHRH polypeptide.

11. The composition of claim 9, wherein the encoded GHRH or functional biological equivalent thereof is of formula (SEQID #6):
X₁-x₂-DAIFTNSYRKVL-X₃-QLSARKLLQDI-X₄-X₅-RQQGERNQEQGA-OH
wherein the formula has the following characteristics:
X₁ is a D-or L-isomer of the amino acid tyrosine (“Y”), or histidine (“H”);
X₂ is a D-or L-isomer of the amino acid alanine (“A”), valine (“V”), or isoleucine (“I”);
X₃ is a D-or L-isomer of the amino acid alanine (“A”) or glycine (“G”);
X₄ is a D-or L-isomer of the amino acid methionine (“M”), or leucine (“L”);
X₅ is a D-or L-isomer of the amino acid serine (“S”) or asparagine (“N”); or a combination thereof.

12. The composition of claim 1, wherein the nucleic acid expression construct encodes a polypeptide of a sequence comprising SeqID#1, SeqID#2, SeqID#3, SeqID#4, or SeqID#5.

13. A composition comprising:
(a) a nucleic acid expression construct; and
(b) a poly-L-glutamate polypeptide associated therewith;
wherein a ratio in moles of the charged transfection-facilitating polypeptide to nucleic acid expression construct comprises from 1 mole to 5,000 moles of the charged transfection-facilitating polypeptide per mole of nucleic acid expression construct.

14. The composition of claim 13, wherein the ratio in moles of the charged transfection-facilitating polypeptide to nucleic acid expression construct is equal to 1,200 moles or less of the charged transfection-facilitating polypeptide per mole of nucleic acid expression construct.

15. The composition of claim 13, wherein the ratio in moles of the charged transfection-facilitating polypeptide to nucleic acid expression construct is equal to 1 mole of the charged transfection-facilitating polypeptide per mole of nucleic acid expression construct.

16. The composition of claim 13, wherein an average molecular length of the nucleic acid expression vector is from about 2,000 to about 5,000 nucleotide base pairs.

17. The composition of claim 13, wherein an average molecular weight of the charged transfection-facilitating polypeptide is from about 400 to about 30,000 Da.

18. The composition of claim 13, wherein an average molecular length of the nucleic acid expression vector is about 5,000 nucleotide base pairs, and an average molecular weight of the charged transfection-facilitating polypeptide is about 10,900 Da.

19. The composition of claim 13, wherein the nucleic acid expression construct comprises SeqID#11, SeqID#12, SeqID#13, SeqID#14, SeqID#17, SeqID#18, SeqID#19, SeqID#20, or SeqID#21.

20. The composition of claim 13, wherein the nucleic acid expression construct comprises a gene that encodes a growth-hormone-releasing-hormone (“GHRH”) or functional biological equivalent thereof.

21. The composition of claim 20, wherein the encoded GHRH is a biologically active polypeptide, and the encoded functional biological equivalent of GHRH is a polypeptide that has been engineered to contain a distinct amino acid sequence while simultaneously having similar or improved biologically activity when compared to the GHRH polypeptide.

22. The composition of claim 20, wherein the encoded GHRH or functional biological equivalent thereof is of formula (SEQID#6):
-X₋₁-X₂-DAIFTNSYRKVL-X₃-QLSARKLLQDI-X₄-X₅-RQQGERNQEQGA-OH
wherein the formula has the following characteristics:
X₁ is a D-or L-isomer of the amino acid tyrosine (“Y”), or histidine (“H”);
X₂ is a D-or L-isomer of the amino acid alanine (“A”), valine (“V”), or isoleucine (“I”);
X₃ is a D-or L-isomer of the amino acid alanine (“A”) or glycine (“G”);
X₄ is a D-or L-isomer of the amino acid methionine (“M”), or leucine (“L”);
X₅ is a D-or L-isomer of the amino acid serine (“S”) or asparagine (“N”); or a combination thereof.

23. The composition of claim 13, wherein the nucleic acid expression construct encodes a polypeptide of a sequence comprising SeqID#1, SeqID#2, SeqID#3, SeqID#4, or SeqID#5.

24. A composition comprising:
(a) a nucleic acid expression construct encoding a growth hormone releasing hormone (“GHRH”) or functional biological equivalent thereof, and
(b) a poly-L-glutamate polypeptide associated therewith,
wherein a ratio in moles of the charged transfection-facilitating polypeptide to nucleic acid expression construct comprises from 1 mole to 5,000 moles of the charged transfection-facilitating polypeptide per mole of nucleic acid expression construct.

25. The composition of claim 24, wherein the ratio in moles of the charged transfection-facilitating polypeptide to nucleic acid expression construct is equal to 1,200 moles or less of the charged transfection-facilitating polypeptide per mole of nucleic acid expression construct.

26. The composition of claim 24, wherein the ratio in moles of the charged transfection-facilitating polypeptide to nucleic acid expression construct is equal to 1 mole of the charged transfection-facilitating polypeptide per mole of nucleic acid expression construct.

27. The composition of claim 24, wherein an average molecular length of the nucleic acid expression vector is from about 2,000 to about 5,000 nucleotide base pairs.

28. The composition of claim 24, wherein an average molecular weight of the charged transfection-facilitating polypeptide is from about 400 to about 30,000 Da.

29. The composition of claim 24, wherein an average molecular length of the nucleic acid expression vector is about 5,000 nucleotide base pairs, and an average molecular weight of the charged transfection-facilitating polypeptide is about 10,900 Da.

30. The composition of claim 24, wherein the nucleic acid expression construct comprises SeqID#11, SeqID#12, SeqID#13, SeqID#14, SeqID#17, SeqID#18, SeqID#19, SeqID#20, or SeqID#21.

31. The composition of claim 24, wherein the encoded GHRH is a biologically active polypeptide, and the encoded functional biological equivalent of GHRH is a polypeptide that has been engineered to contain a distinct amino acid sequence while simultaneously having similar or improved biologically activity when compared to the GHRH polypeptide.

32. The composition of claim 24, wherein the encoded GHRH or functional biological equivalent thereof is of formula (SEQID#6):
-X₋₁-X₂-DAIFTNSYRKVL-X₃-QLSARKLLQDI-X₄-X₅-RQQGERNQEQGA-OH
wherein the formula has the following characteristics:
X₁ is a D-or L-isomer of the amino acid tyrosine (“Y”), or histidine (“H”);
X₂ is a D-or L-isomer of the amino acid alanine (“A”), valine (“V”), or isoleucine (“I”);
X₃ is a D-or L-isomer of the amino acid alanine (“A”) or glycine (“G”);
X₄ is a D-or L-isomer of the amino acid methionine (“M”), or leucine (“L”);
X₅ is a D-or L-isomer of the amino acid serine (“S”) or asparagine (“N”); or a combination thereof.

33. The method of claim 24, wherein the nucleic acid expression construct encodes a polypeptide of a sequence comprising SeqID#1, SeqID#2, SeqID#3, SeqID#4, or SeqID#5.

34. A composition comprising:
(a) a nucleic acid expression construct encoding a growth hormone releasing hormone (“GHRH”) or functional biological equivalent thereof, and
(b) a charged transfection-facilitating polypeptide associated therewith;
wherein a ratio in moles of the charged transfection-facilitating polypeptide to nucleic acid expression construct comprises from 1 mole to 5,000 moles of the charged transfection-facilitating polypeptide per mole of nucleic acid expression construct.

35. The composition of claim 34, wherein the ratio in moles of the charged transfection-facilitating polypeptide to nucleic acid expression construct is equal to 1,200 moles or less of the charged transfection-facilitating polypeptide per mole of nucleic acid expression construct.

36. The composition of claim 34, wherein the ratio in moles of the charged transfection-facilitating polypeptide to nucleic acid expression construct is equal to 1 mole of the charged transfection-facilitating polypeptide per mole of nucleic acid expression construct.

37. The composition of claim 34, wherein an average molecular length of the nucleic acid expression vector is from about 2,000 to about 5,000 nucleotide base pairs.

38. The composition of claim 34, wherein an average molecular weight of the charged transfection-facilitating polypeptide is from about 400 to about 30,000 Da.

39. The composition of claim 34, wherein an average molecular length of the nucleic acid expression vector is about 5,000 nucleotide base pairs, and an average molecular weight of the charged transfection-facilitating polypeptide is about 10,900 Da.

40. The composition of claim 34, wherein the charged transfection-facilitating polypeptide comprises poly-L-glutamate.

41. The composition of claim 34, wherein the nucleic acid expression construct comprises SeqID#11, SeqID#12, SeqID#13, SeqID#14, SeqID#17, SeqID#18, SeqID#19, SeqID#20, or SeqID#21.

42. The composition of claim 34, wherein the encoded GHRH is a biologically active polypeptide, and the encoded functional biological equivalent of GHRH is a polypeptide that has been engineered to contain a distinct amino acid sequence while simultaneously having similar or improved biologically activity when compared to the GHRH polypeptide.

43. The composition of claim 34, wherein the encoded GHRH or functional biological equivalent thereof is of formula (SEQID#6):
-X₋₁-X₂-DAIFTNSYRKVL-X₃-QLSARKLLQDI-X₄-X₅-RQQGERNQEQGA-OH
wherein the formula has the following characteristics:
X₁ is a D-or L-isomer of the amino acid tyrosine (“Y”), or histidine (“H”);
X₂ is a D-or L-isomer of the amino acid alanine (“A”), valine (“V”), or isoleucine (“I”);
X₃ is a D-or L-isomer of the amino acid alanine (“A”) or glycine (“G”);
X₄ is a D-or L-isomer of the amino acid methionine (“M”), or leucine (“L”);
X₅ is a D-or L-isomer of the amino acid serine (“S”) or asparagine (“N”); or a combination thereof.

44. The method of claim 34, wherein the nucleic acid expression construct encodes a polypeptide of a sequence comprising SeqID#1, SeqID#2, SeqID#3, SeqID#4, or SeqID#5.

45. A method for introducing a nucleic acid expression construct into a cell of a selected tissue in a recipient, comprising:
(a) placing a plurality of electrodes in the selected tissue, wherein the plurality of electrodes are arranged in a spaced relationship;
(b) introducing the nucleic acid expression construct having a charged transfection-facilitating polypeptide associated therewith; and
(c) applying a constant current electrical pulse to the plurality of electrodes;
wherein a ratio in moles of the charged transfection-facilitating polypeptide to nucleic acid expression construct comprises from 1 mole to 5,000 moles of the charged transfection-facilitating polypeptide per mole of nucleic acid expression construct.

46. The composition of claim 45, wherein the nucleic acid expression construct comprises SeqID#11, SeqID#12, SeqID#13, SeqID#14, SeqID#17, SeqID#18, SeqID#19, SeqID#20, or SeqID#21.

47. The method of claim 45, wherein the cell of the selected tissue comprises a somatic cell, a stem cell, or a germ cell.

48. The method of claim 45, wherein the selected tissue in the recipient comprises muscle.

49. The method of claim 45, wherein the charged transfection-facilitating polypeptide comprises poly-L-glutamate.

50. The method of claim 45, wherein a ratio in moles of the charged transfection-facilitating polypeptide to nucleic acid expression construct is equal to 1,200 moles or less of the charged transfection-facilitating polypeptide per mole of nucleic acid expression construct.

51. The method of claim 45, wherein a ratio in moles of the charged transfection-facilitating polypeptide to nucleic acid expression construct is equal to 1 mole of the charged transfection-facilitating polypeptide per mole of nucleic acid expression construct.

52. The method of claim 45, wherein the plurality of electrodes are constructed from a material that will make galvanic contact with the tissues.

53. The method of claim 45, wherein the nucleic acid expression construct comprises a gene that encodes a growth-hormone-releasing-hormone (“GHRH”) or functional biological equivalent thereof.

54. The method of claim 53, wherein the encoded GHRH or functional biological equivalent thereof is expressed in a tissue specific cell of the subject.

55. The method of claim 53, wherein the encoded GHRH is a biologically active polypeptide; and the encoded functional biological equivalent of GHRH is a polypeptide that has been engineered to contain a distinct amino acid sequence while simultaneously having similar or improved biologically activity when compared to the GHRH polypeptide.

56. The method of claim 53, wherein the encoded GHRH or functional biological equivalent thereof is of formula (SEQID#6):
-X₋₁-X₂-DAIFTNSYRKVL-X₃-QLSARKLLQDI-X₄-X₅-RQQGERNQEQGA-OH
wherein the formula has the following characteristics:
X₁ is a D-or L-isomer of the amino acid tyrosine (“Y”), or histidine (“H”);
X₂ is a D-or L-isomer of the amino acid alanine (“A”), valine (“V”), or isoleucine (“I”);
X₃ is a D-or L-isomer of the amino acid alanine (“A”) or glycine (“G”);
X₄ is a D-or L-isomer of the amino acid methionine (“M”), or leucine (“L”);
X₅ is a D-or L-isomer of the amino acid serine (“S”) or asparagine (“N”); or a combination thereof.

57. The method of claim 45, wherein the nucleic acid expression construct encodes a polypeptide of a sequence comprising SeqID#1, SeqID#2, SeqID#3, SeqID#4, or SeqID#5.

58. A method for introducing a nucleic acid expression construct into a muscle cell in a body, comprising:
(a) placing a plurality of electrodes in the selected tissue, wherein the plurality of electrodes are arranged in a spaced relationship;
(b) introducing the nucleic acid expression construct having a charged transfection-facilitating polypeptide associated therewith; wherein charged transfection-facilitating polypeptide comprises a poly-L-glutamate polypeptide;
(c) applying an electrical pulse to the plurality of electrodes,
wherein the nucleic acid expression construct encodes a growth hormone releasing hormone (“GHRH”) or functional biological equivalent thereof; and a ratio in moles of the charged transfection-facilitating polypeptide to nucleic acid expression construct comprises from 1 mole to 5,000 moles of the charged transfection-facilitating polypeptide per mole of nucleic acid expression construct.

59. The method of claim 58, wherein an average molecular length of the nucleic acid expression vector is from about 2,000 to about 5,000 nucleotide base pairs.

60. The method of claim 58, wherein an average molecular weight of the charged transfection-facilitating polypeptide is from about 400 to about 30,000 Da.

61. The method of claim 58, wherein an average molecular length of the nucleic acid expression vector is about 5,000 nucleotide base pairs, and an average molecular weight of the charged transfection-facilitating polypeptide is about 10,900 Da.

62. The method of claim 58, wherein the nucleic acid expression construct comprises SeqID#11, SeqID#12, SeqID#13, SeqID#14, SeqID#17, SeqID#18, SeqID#19, SeqID#20, or SeqID#21.

63. The method of claim 58, wherein a ratio in moles of the charged transfection-facilitating polypeptide to nucleic acid expression construct is equal to 1,200 moles or less of the charged transfection-facilitating polypeptide per mole of nucleic acid expression construct.

64. The method of claim 58, wherein a ratio in moles of the charged transfection-facilitating polypeptide to nucleic acid expression construct is equal to 1 mole of the charged transfection-facilitating polypeptide per mole of nucleic acid expression construct.

65. The method of claim 58, wherein the plurality of needle electrodes are constructed from a material that will make galvanic contact with the tissues.

66. The method of claim 58, wherein introducing the nucleic acid expression construct into the muscle cell of the recipient initiates expression of an encoded GHRH or functional biological equivalent thereof.

67. The method of claim 58, wherein the encoded GHRH or functional biological equivalent thereof is expressed in a tissue specific cell of the subject.

68. The method of claim 58, wherein the encoded GHRH is a biologically active polypeptide; and the encoded functional biological equivalent of GHRH is a polypeptide that has been engineered to contain a distinct amino acid sequence while simultaneously having similar or improved biologically activity when compared to the GHRH polypeptide.

69. The method of claim 58, wherein the encoded GHRH or functional biological equivalent thereof is of formula (SEQID#6):
-X₋₁-X₂-DAIFTNSYRKVL-X₃-QLSARKLLQDI-X₄-X₅-RQQGERNQEQGA-OH
wherein the formula has the following characteristics:
X₁ is a D-or L-isomer of the amino acid tyrosine (“Y”), or histidine (“H”);
X₂ is a D-or L-isomer of the amino acid alanine (“A”), valine (“V”), or isoleucine (“I”);
X₃ is a D-or L-isomer of the amino acid alanine (“A”) or glycine (“G”);
X₄ is a D-or L-isomer of the amino acid methionine (“M”), or leucine (“L”);
X₅ is a D-or L-isomer of the amino acid serine (“S”) or asparagine (“N”); or a combination thereof.

70. The method of claim 58, wherein the nucleic acid expression construct encodes a polypeptide of a sequence comprising SeqID#1, SeqID#2, SeqID#3, SeqID#4, or SeqID#5.

71. A method to increase stability of a nucleic acid expression construct, comprising: mixing the nucleic acid expression construct with a charged transfection-facilitating polypeptide to give a stabilized nucleic acid expression construct;
wherein
(a) the in vitro degradation of the stabilized nucleic acid expression construct is slower as compared to that of the nucleic acid expression construct not associated with a transfection-facilitation polypeptide; and
(b) a ratio in moles of the charged transfection-facilitating polypeptide to nucleic acid expression construct comprises from 1 mole to 5,000 moles of the charged transfection-facilitating polypeptide per mole of nucleic acid expression construct.

72. The method of claim 71, wherein charged transfection-facilitating polypeptide comprises a poly-L-glutamate polypeptide.

73. The method of claim 71, wherein an average molecular length of the nucleic acid expression vector is from about 2,000 to about 5,000 nucleotide base pairs.

74. The method of claim 71, wherein an average molecular weight of the charged transfection-facilitating polypeptide is from about 400 to about 30,000 Da.

75. The method of claim 71, wherein an average molecular length of the nucleic acid expression vector is about 5,000 nucleotide base pairs, and an average molecular weight of the charged transfection-facilitating polypeptide is about 10,900 Da.

76. The method of claim 71, wherein the nucleic acid expression construct comprises SeqID#11, SeqID#12, SeqID#13, SeqID#14, SeqID#17, SeqID#18, SeqID#19, SeqID#20, or SeqID#21.

77. The method of claim 71, wherein a ratio in moles of the charged transfection-facilitating polypeptide to nucleic acid expression construct is equal to 1,200 moles or less of the charged transfection-facilitating polypeptide per mole of nucleic acid expression construct.

78. The method of claim 71, wherein a ratio in moles of the charged transfection-facilitating polypeptide to nucleic acid expression construct is equal to 1 mole of the charged transfection-facilitating polypeptide per mole of nucleic acid expression construct.

79. The method of claim 71, wherein the nucleic acid expression construct encodes a growth hormone releasing hormone (“GHRH”) or functional biological equivalent thereof.

80. The method of claim 79, wherein the encoded GHRH or functional biological equivalent thereof is of formula (SEQID#6):
-X₋₁-X₂-DAIFTNSYRKVL-X₃-QLSARKLLQDI-X₄-X₅-RQQGERNQEQGA-OH
wherein the formula has the following characteristics:
X₁ is a D-or L-isomer of the amino acid tyrosine (“Y”), or histidine (“H”);
X₂ is a D-or L-isomer of the amino acid alanine (“A”), valine (“V”), or isoleucine (“I”);
X₃ is a D-or L-isomer of the amino acid alanine (“A”) or glycine (“G”);
X₄ is a D-or L-isomer of the amino acid methionine (“M”), or leucine (“L”);
X₅ is a D-or L-isomer of the amino acid serine (“S”) or asparagine (“N”); or a combination thereof.

81. The method of claim 71, wherein the nucleic acid expression construct encodes a polypeptide of a sequence comprising SeqID#1, SeqID#2, SeqID#3, SeqID#4, or SeqID#5.

82. A method to increase stability of a nucleic acid expression construct, comprising: mixing the nucleic acid expression construct with a charged transfection-facilitating polypeptide to give a stabilized nucleic acid expression construct ps wherein
the in vitro degradation of the stabilized nucleic acid expression construct is slower as compared to that of the nucleic acid expression construct not associated with a transfection-facilitation polypeptide;
the charged transfection-facilitating polypeptide comprises a poly-L-glutamate polypeptide;
the nucleic acid expression construct encodes a growth hormone releasing hormone (“GHRH”) or functional biological equivalent thereof; and
a ratio in moles of the charged transfection-facilitating polypeptide to nucleic acid expression construct comprises from 1 mole to 5,000 moles of the charged transfection-facilitating polypeptide per mole of nucleic acid expression construct.

83. The method of claim 82, wherein an average molecular length of the nucleic acid expression vector is from about 2,000 to about 5,000 nucleotide base pairs.

84. The method of claim 82, wherein an average molecular weight of the charged transfection-facilitating polypeptide is from about 400 to about 30,000 Da.

85. The method of claim 82, wherein an average molecular length of the nucleic acid expression vector is about 5,000 nucleotide base pairs, and an average molecular weight of the charged transfection-facilitating polypeptide is about 10,900 Da.

86. The method of claim 82, wherein the nucleic acid expression construct comprises SeqID#11, SeqID#12, SeqID#13, SeqID#14, SeqID#17, SeqID#18, SeqID#19, SeqID#20, or SeqID#21.

87. The method of claim 82, wherein the ratio in moles of the charged transfection-facilitating polypeptide to nucleic acid expression construct is equal to 1,200 moles or less of the charged transfection-facilitating polypeptide per mole of nucleic acid expression construct.

88. The method of claim 82, wherein the ratio in moles of the charged transfection-facilitating polypeptide to nucleic acid expression construct is equal to 1 mole of the charged transfection-facilitating polypeptide per mole of nucleic acid expression construct.

89. The method of claim 82, wherein the encoded GHRH or functional biological equivalent thereof is of formula (SEQID#6):
-X₋₁-X₂-DAIFTNSYRKVL-X₃-QLSARKLLQDI-X₄-X₅-RQQGERNQEQGA-OH
wherein the formula has the following characteristics:
X₁ is a D-or L-isomer of the amino acid tyrosine (“Y”), or histidine (“H”);
X₂ is a D-or L-isomer of the amino acid alanine (“A”), valine (“V”), or isoleucine (“I”);
X₃ is a D-or L-isomer of the amino acid alanine (“A”) or glycine (“G”);
X₄ is a D-or L-isomer of the amino acid methionine (“M”), or leucine (“L”);
X₅ is a D-or L-isomer of the amino acid serine (“S”) or asparagine (“N”); or a combination thereof.

90. The method of claim 82, wherein the nucleic acid expression construct encodes a polypeptide of a sequence comprising SeqID#1, SeqID#2, SeqID#3, SeqID#4, or SeqID#5.
